(19) United States
(12) Patent Application Publication
(10) Pub. No.: US 2009/0285821 A1
(43) Pub. Date: Nov. 19, 2009

(54) Campylobacter Pilus Protein, Compositions and Methods

(75) Inventors: Lynn Joens, Tucson, AZ (US); James R. Theoret, Maricopa, AZ (US); Ryan J. Reeser, Tucson, AZ (US); Bibiana Law, Tucson, AZ (US)

Correspondence Address: GREENLEE WINNER AND SULLIVAN P C, 4875 PEARL EAST CIRCLE, SUITE 200, BOULDER, CO, 80301, US

(73) Assignee: THE ARIZONA BOARD OF REGENTS ON BEHALF OF THE UNIVERSITY OF ARIZONA, TUCSON, AZ (US)

(21) Appl. No.: 12/373,274

(22) PCT Filed: Dec. 1, 2006
(86) PCT No.: PCT/US06/61470

§ 371 Date: Apr. 20, 2009

Related U.S. Application Data
(60) Provisional application No. 60/819,589, filed on Jul. 10, 2006.

(51) Int. Cl. A61K 39/395; C12N 15/11; C12N 15/00; C12N 1/21; A61K 39/02; C07K 16/12; C12P 21/02; G01N 33/569

(52) U.S. Cl. 424/139.1; 536/23.7; 435/320.1; 435/252.3; 424/190.1; 530/387.9; 435/69.3; 435/7.32

(57) ABSTRACT

The present disclosure provides coding and amino acid sequences for a Campylobacter jejuni pilus protein (and from other species as well). This protein, when administered to a human or animal, elicits the expression of an immune response to Campylobacter jejuni, with the result that colonization and/or infection by this organism is reduced. Recombinant protein or biofilm material comprising the pilus protein is formulated into immunogenic compositions, especially for mucosal administration. Thus, the present invention provides methods for improvement of the microbial quality of food products including poultry, eggs, meat and dairy products, and indirectly of plant foods that may come in contact with agricultural waste, either from fertilization or from irrigation water.

### CROSS-REFERENCE TO RELATED APPLICATIONS

This application claims the benefit of U.S. Provisional Application No. 60/819,589, filed Jul. 10, 2006, which application is incorporated by reference herein to the extent there is no inconsistency with the present disclosure.

### STATEMENT REGARDING FEDERALLY SPONSORED RESEARCH OR DEVELOPMENT

This invention was made with government support under USDA/CSREES Grant No. 2005-51110-02333 awarded by the United States Department of Agriculture. The government has certain rights in the invention.

### REFERENCE TO SEQUENCE LISTING, A TABLE, OR A COMPUTER PROGRAM LISTING COMPACT DISK APPENDIX

The Sequence Listing filed on even date herewith is incorporated by reference herein.

### BACKGROUND OF THE INVENTION

The field of this invention is the area of immunogenic compositions, methods, vaccines and genes encoding bacterial virulence determinants, particularly those genes encoding a pilus protein of Campylobacter jejuni or other species of Campylobacter and those immunogenic compositions comprising such a pilus protein.

C. jejuni is a gram negative, curved to spiral rod with polar flagella and grows best in a microaerophilic environment ranging from 37° C. to 42° C. (4; 7; 12; 23; 26). In the U.S. it is estimated that approximately 2.1 to 2.4 million cases of campylobacteriosis occur annually with a cost of $8 billion (16; 17).

Campylobacteriosis can either be asymptomatic or result in a variety of symptoms. In developing countries, infection may be asymptomatic or it may result in relatively mild diarrhea. In industrialized countries campylobacterial infections present as self-limiting gastrointestinal infections characterized by diarrhea with or without blood or mucus, vomiting, cramping and fever. Symptomatic infections consist of an acute onset of watery diarrhea, abdominal pain, fever, and the presence of blood and leukocytes in stool samples and are usually self limiting, lasting from two to 11 days, but in immunocompromised individuals infections can persist for greater that 3 months (4; 6; 16). Long term secondary effects of infection may include reactive arthritis, Reiters syndrome, ophthalmitis in HLA B 27 positive patients and Guillain Barre syndrome (15; 18).

Campylobacters are considered normal flora of the gut of a number of domestic animals and birds (1; 2; 5; 8; 31). The ability of these birds to shed Campylobacter can cause contamination of waterways or water systems, and thus acting as a source of contamination for other animals or humans. Campylobacter infections occur through oral routes including; ingestion of contaminated water, unpasteurized milk or cheese, consuming undercooked or raw foods such as poultry (5; 8; 31). However, consumption of raw milk and undercooked poultry is the major sources of Campylobacter infections. The ability of C. jejuni to form biofilms and becoming a continual source of inoculum for domesticated animals and humans has also been the subject of other studies (8; 31). C. jejuni has the ability to form biofilms in the watering supplies and plumbing systems of animal husbandry facilities and animal processing plants, thus becoming a source of infection and contamination (8; 31). However, this possibility is supported by a very limited number of publications showing that C. jejuni can form biofilms on abiotic surfaces.

Because of the health costs, there is a need in the art for vaccines effective for reducing the colonization of poultry and/or cattle with C. jejuni and for reducing the incidence of C. jejuni infections.

### BRIEF SUMMARY OF THE INVENTION

An object of the present invention is to provide a nucleotide sequence encoding a pilus protein from Campylobacter jejuni. As specifically exemplified, the encoded pilus protein has a coding sequence as given in SEQ ID NO:1. The encoded pilus protein has an amino acid sequence as given in SEQ ID NO:2. Coding sequences and amino acid sequences with at least 70% sequence identity to the specifically exemplified sequences are within the scope of the present invention.

It is an additional object of the invention to provide non-naturally occurring (“recombinant”) nucleic acid molecules for the recombinant production of the C. jejuni pilus protein of the present invention and methods for recombinantly producing this protein.

The skilled artisan understands that the coding sequence and amino acid sequence of the exemplified pilus protein can be used to identify and isolate additional, nonexemplified nucleotide sequences which will encode a protein of the same amino acid sequence as given in SEQ ID NO:2, or an amino acid sequence of greater than 70%, 80%, 85%, 90%, 95% (and all integer percents between 70 and 100) identity thereto and having equivalent biological activity. When it is desired that the sequence encoding a pilus protein of the present invention be expressed, then the skilled artisan operably links transcription and translational control regulatory sequences to the coding sequence, with the choice of the regulatory sequences being determined by the host cell in which the coding sequence is to be expressed. With respect to a recombinant DNA molecule carrying a C. jejuni pilus protein coding sequence, the skilled artisan can choose a vector (such as a plasmid or a viral vector) which can be introduced into and which can replicate in the host cell. The host cell can be a bacterium, preferably Escherichia coli or a nonvirulent Salmonella typhimurium or, alternatively, a yeast or mammalian cell.

In another embodiment, recombinant polynucleotides which encode a pilus protein including, e.g., protein fusions or deletions, as well as expression systems are provided. Expression systems are defined as polynucleotides which, when transformed into an appropriate host cell, can express the pilus protein of the present invention or a functionally equivalent protein. The recombinant polynucleotides possess a nucleotide sequence which is substantially similar to a natural C. jejuni pilus protein-encoding polynucleotide or a fragment thereof. Expression may be under the control of the promoter normally associated with the gene or the pilus protein coding sequence can be expressed under the regulatory control of a heterologous promoter (one not in nature associated with the pilus protein coding sequence). The preferred enteric bacterial host strain for pilus protein production is a strain of E. coli.

Further provided by the present invention are oligonucleotides and polynucleotides which are capable of hybridizing specifically, using standard conditions well understood by the skilled artisan, to C. jejuni genomic DNA, cloned DNA (or to the cognate mRNA) encoding the pilus protein of the present invention. These pilus protein-specific sequences can also be used in the preparation of primers for use in the polymerase chain reaction (PCR) for the amplification of pilus protein encoding nucleic acid. Either hybridization or PCR can be adapted for use in the detection of C. jejuni in biological, food, cheese, water, fecal or environmental samples or in the diagnosis of disease caused by C. jejuni.

The polynucleotides include RNA, cDNA, genomic DNA, synthetic forms, and mixed polymers, both sense and antisense strands, and may be chemically or biochemically modified or contain non-natural or derivatized nucleotide bases. DNA is preferred. Recombinant polynucleotides comprising sequences otherwise not naturally occurring are also provided by this invention, as are alterations of a wild type C. jejuni pilus protein encoding sequence, including but not limited to deletion, insertion, substitution of one or more nucleotides or by fusion to other polynucleotide sequences, provided that such changes in the primary sequence of the pilus protein do not alter the epitope(s) capable of eliciting protective immunity.

The present invention also provides for fusion polypeptides comprising a C. jejuni pilus protein or an antigenic portion thereof. Heterologous fusions may be constructed which would exhibit a combination of properties or activities of the proteins from which they are derived. Potential fusion partners include, but are not limited to, immunoglobulins, ubiquitin, bacterial β-galactosidase, trpE, protein A, β-lactamase, alpha amylase, alcohol dehydrogenase and yeast alpha mating factor (Godowski et al. (1988) Science, 241, 812-816) or various protein “tag” sequences (flagellar antigen, poly-histidine, streptavidin, glutathione S-transferase, among others known to the art). Fusion proteins are typically made by recombinant methods but may be chemically synthesized as well understood in the art.

Compositions and immunogenic preparations including, but not limited to, vaccines comprising naturally expressed C. jejuni pili or recombinant C. jejuni pilus protein and a suitable carrier are provided by the present invention, and these compositions and preparations can advantageously further comprise at least one adjuvant. Also encompassed by the present invention are immunogenic compositions comprising pilus-expressing live attenuated Campylobacter with expressed pilus protein (for example, in a biofilm) or killed cell biofilm material which contains the pilus protein. Such compositions are useful, for example, in immunizing a human against diarrheal disease resulting from infection by C. jejuni or in reducing the incidence of C. jejuni in poultry or bovines, so as to reduce contamination in food products and in the environment. The preparations comprise an immunogenic amount of a pilus protein of the present invention or an immunogenic fragment thereof. Such immunogenic compositions can advantageously further comprise pilus proteins or antigenic determinants therefrom of one or more other serological types or other antigenic materials derived from C. jejuni. By “immunogenic amount” is meant an amount capable of eliciting the production of antibodies, preferably conferring protective immunity directed against colonization or infection by C. jejuni with the same pilus protein or an immunologically cross reactive pilus protein as in the immunogenic composition. The immunogenic compositions can further comprise an adjuvant, for example, cholera toxin or a cholera toxin subunit B for compositions designed for mucosal administration.

Also within the scope of the present invention are methods for reducing the incidence of C. jejuni in poultry or in beef or dairy cattle by administering an immunogenic composition comprising the C. jejuni pilus protein as taught herein to the poultry or cattle. The route of administration can be mucosal (especially nasal or oral) or it can be subcutaneous, intramuscular, intradermal, intraperitoneal or parenteral. Similarly, human incidence of Campylobacter infection and disease can be reduced by administering such an immunogenic composition to a human in need thereof, preferably by mucosal administration.

The present invention further provides antibody which specifically binds the pilus protein and methods for detecting C. jejuni, especially in biofilms and/or food products, or for diagnosing Campylobacter infection. In addition, the pilus protein can be used in methods for monitoring an immune response or for detecting antibodies to it, for example, to assess exposure to a Campylobacter or to follow the development of an immune response.

### BRIEF DESCRIPTION OF THE DRAWINGS

FIG. 1A illustrates the effects of growth media on C. jejuni biofilm formation. MHB, Brucella and Bolton broths were assessed for their ability to promote C. jejuni M129 biofilm formation as measured by CV staining. Experiments were performed in triplicate on three separate occasions. Error bars represent one standard deviation from mean.

FIG. 1B shows the effects of temperature and O₂ tension on C. jejuni M129 biofilm formation as measured by Crystal Violet (CV) staining. Growth conditions, which were favorable for the growth of C. jejuni M129, such as 37° C. and 10% CO₂ resulted in better biofilm formation. Experiments were performed three times in triplicate. Error bars represent one standard deviation from mean.

FIG. 2A shows the effect of sucrose or glucose and FIG. 2B shows the effect of NaCl on the ability of C. jejuni M129 to form biofilms as measured by CV staining. In FIG. 2A white bars represent sucrose, grey bars represent glucose. FIG. 2B shows the extent of biofilm formation at different concentrations of NaCl. Experiments were performed three times in triplicate. Error bars represent one standard deviation from mean.

FIG. 3 shows that C. jejuni M129 forms biofilms on abiotic surfaces as measured by CV staining. Experiments were performed three times in triplicate. Error bars represent one standard deviation from mean.

FIG. 4 shows that chloramphenicol inhibits biofilm formation of C. jejuni M129 and F38011. C. jejuni strains were treated with 0.5 μg/ml chloramphenicol for 15 minutes prior to assaying for biofilm formation in the absence of antibiotic. Experiments were performed three times in triplicate. Error bars represent one standard deviation from mean.

FIGS. 5A and 5B show that the presence of C. jejuni M129 flagella positively influences biofilm formation. FIG. 5A: Biofilm formation as a function of CV staining. Experiments were performed three times in triplicate. Error bars represent one standard deviation from mean. FIG. 5B: Representative wells shown at 24, 48, and 72 hr post incubation, showing CV staining prior to decolorization.

FIGS. 5C and 5D show that the C. jejuni M129 quorum sensing ability positively influences biofilm formation. FIG. 5C: Biofilm formation as measured by CV staining. Experiments were performed three times in triplicate. Error bars represent one standard deviation from mean. FIG. 5D: Representative wells shown at 24, 48, and 72 hr post incubation, showing CV staining prior to decolorization.

FIG. 6 shows that culture supernatant fluids from Gram negative and Gram positive bacteria can influence the formation of C. jejuni M129 biofilms. CSFs from Pseudomonas spp. and A. pyogenes promote C. jejuni biofilm formation. Biofilm assays were performed with C. jejuni M129 in 1:1 mixtures of MHB and CSF from P. aeruginosa 9027, P. fluorescens PF 5, Chromobacterium violaceum CV206, A. pyogenes BBR1 or C. perfringens strain 13. TSB supplemented with 5% newborn calf serum (TSB 5%) and TSB supplemented with 0.5% yeast extract and 0.05% cysteine (TSBYC) were used as controls for A. pyogenes and C. perfringens CSFs, respectively. Experiments were performed three times in triplicate and error bars represent the standard deviation from the mean.

FIG. 7 shows that C. jejuni isolates form biofilms on abiotic surfaces. Biofilm formation by C. jejuni isolates does not correlate with virulence. Experiments were performed three times in triplicate. Error bars represent the standard deviation from mean.

### DETAILED DESCRIPTION OF THE INVENTION

While studying the ability of C. jejuni to form biofilms, it was discovered that these bacteria produced nonpolar pili, in addition to polar flagella. The pili were produced in a non-flagellate flaAB double mutant, as well in wild type strains.

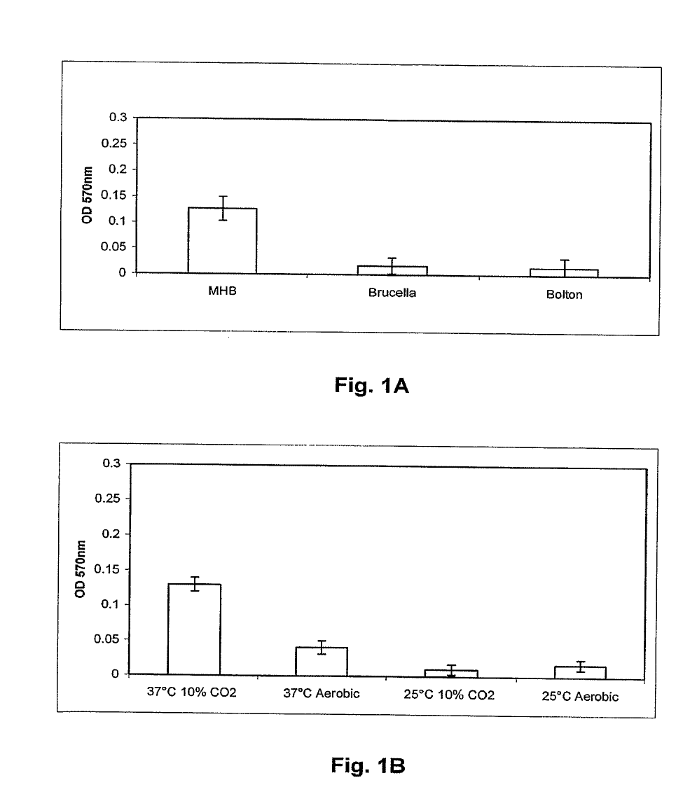

Since environmental factors and media components can affect biofilm formation, we tested various bathing medium conditions for their effects on C. jejuni M129 biofilm formation. MHB, Brucella and Bolton broths were assessed for their ability to promote biofilm formation. The more nutrient rich Brucella and Bolton broths did not support optimal biofilm formation, while significant biofilm formation occurred in the relatively nutrient poor MHB (FIG. 1A).

Incubation temperatures and oxygen tension can also influence biofilm formation (FIG. 1B). C. jejuni biofilm formation was assayed at different combinations of temperatures and oxygenation. The influence of aerobiosis and temperature had dramatic effects on the biofilm formation. As anticipated, biofilm formation was decreased with growth conditions that are less favorable.

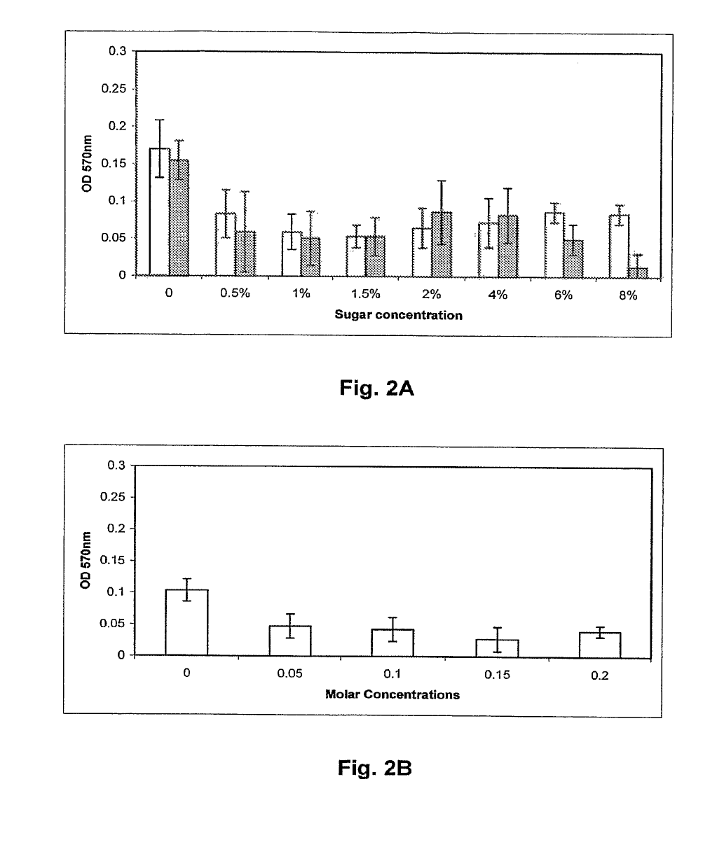

The response of C. jejuni to the osmolytes glucose, sucrose and NaCl with respect to the ability to influence biofilm formation was also examined. As shown in FIGS. 2A and 2B, the presence of glucose, sucrose or NaCl, at all concentrations tested, resulted in significantly decreased biofilm formation. Thus, environmental conditions, as well as medium components, influence the ability of C. jejuni to form biofilms.

Biofilm formation on abiotic surfaces was studied. C. jejuni M129 cells formed biofilms on different hydrophilic materials such as glass and copper and hydrophobic materials such as plastics including polystyrene, polypropylene, polycarbonate, ABS and PVC in varying amounts. To further study C. jejuni M129's ability to form biofilms, materials commonly used in watering systems such as ABS, PVC, copper and a control material polystyrene were quantitatively assayed for their promotion of biofilm formation (FIG. 3). It appeared that the physicochemical properties of the materials may affect attachment and biofilm formation by C. jejuni. C. jejuni cells more readily attached to and formed biofilms on hydrophobic surfaces, albeit to varying degrees. Notably, C. jejuni showed a reduction in biofilm formation on the hydrophilic material copper.

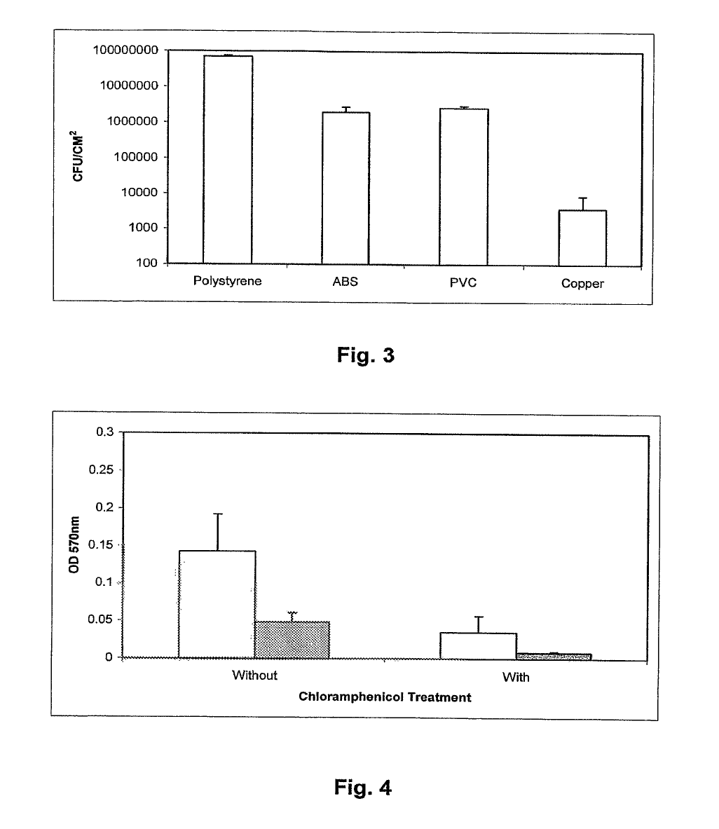

Protein synthesis is required for biofilm formation. Gene expression studies indicate that there is a difference in the profile of proteins synthesized by planktonic (suspended) versus biofilm grown cells suggesting that de novo synthesis of certain proteins maybe required for biofilm formation (9; 10; 19). Protein synthesis inhibitors markedly decrease biofilm formation and can also cause the release of attached bacteria from a biofilm. Therefore, without wishing to be bound by any particular theory, we hypothesized that C. jejuni grown in a biofilm synthesize proteins necessary for this growth phase under specific conditions. When C. jejuni cells were incubated in the presence or absence of chloramphenicol (CM; 0.5 μg/ml) a difference in biofilm formation was observed between the cells treated with CM compared with the untreated control (FIG. 4). These results indicate that C. jejuni cells synthesize proteins required for attachment and biofilm formation in response to appropriate signals and growth conditions.

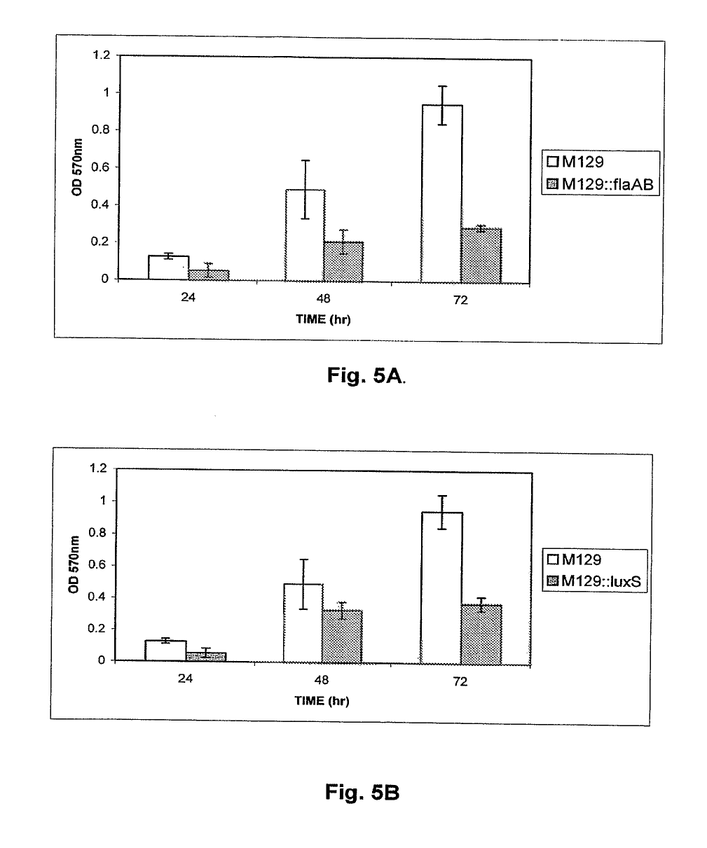

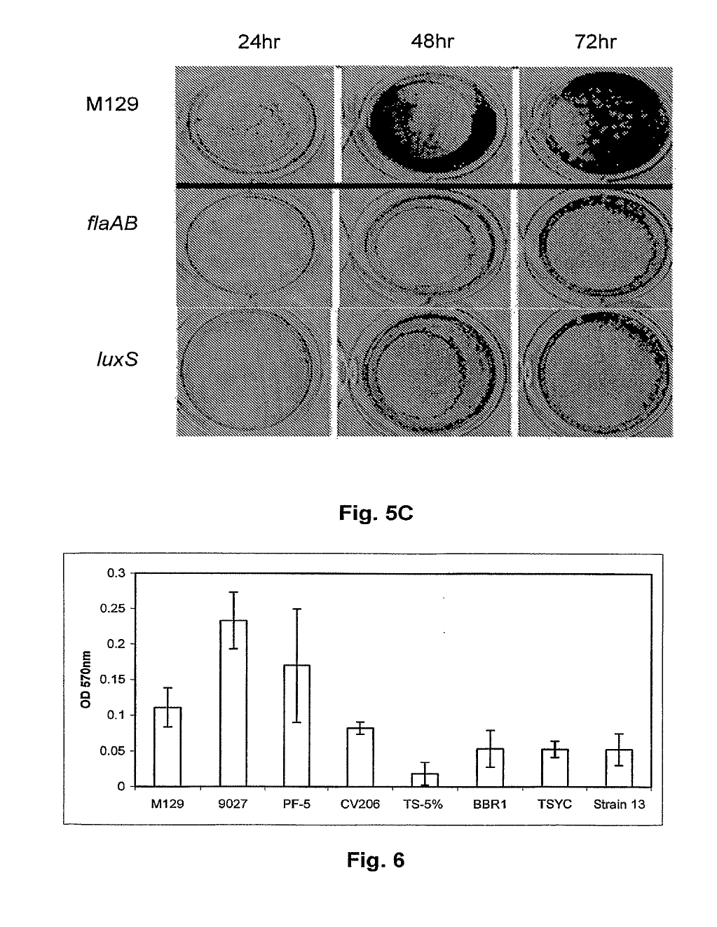

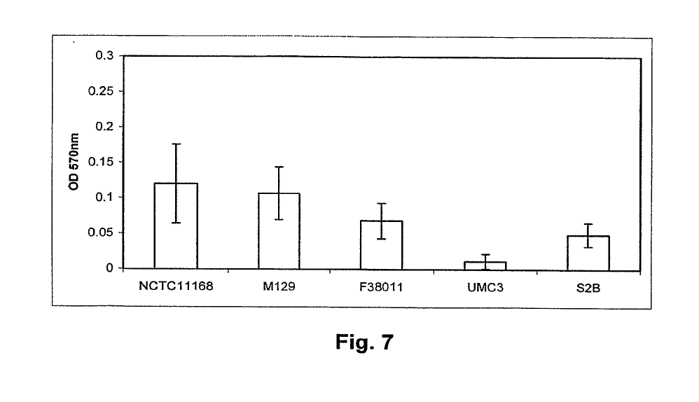

Flagella have been shown to have a role in biofilm formation and affect the rate of attachment by overcoming forces associated with the surface (12; 14; 21; 22). In this study, C. jejuni flagellum minus mutant (M129::flaAB) was assayed for its ability to form biofilms. M129::flaAB showed a slight reduction in biofilm formation compared to that of wild type at 24 hr, but at 48 and 72 hr time points biofilm formation was markedly decreased (FIGS. 5A-5D). We also directly assessed the ability of the flaAB mutant and wildtype M129 to attach to a polystyrene surface using light microscopy (FIG. 5C-D). FIG. 5B shows an increase of adherent cells over a 24, 48 and 72 hr time period for the wildtype M129. However, in the flagella knockout we observed very few cells adherent to the surface at 48 and 72 hr compared to wild type. This data indicates the importance of flagella in C. jejuni biofilm formation.

luxS and quorum sensing signals may influences biofilm formation. To assay the possibility that quorum sensing plays a role in C. jejuni biofilms, a mutation was introduced into the luxS quorum sensing gene with the result that the autoinducer 2 (Al 2) was not produced in mutant cells (11). During biofilm development Al 2 played a significant role in C. jejuni biofilm development as at 48 and 72 hr time points, there was a reduction in biofilm formation for the luxS mutant compared with the wild type C. jejuni (See FIG. 5A-5D).

Due to the close proximity of cells in a biofilm, the biofilm is an ideal environment for quorum sensing to occur (3; 32). During this study, CSFs from various gram negative and gram positive bacteria grown in appropriate media were collected from conditions favorable for biofilm development. C. jejuni isolate M129 was grown in the presence of these culture supernatant fluids, some were believed to contain quorum sensing signals or autoinducers. In the presence of Pseudomonas spp. and Arcanobacterium pyogenes BBR1, culture supernatant fluids cause an increase in biofilm development was observed, but with the other culture supernatant fluids collected, there was no apparent change in the biofilm development (FIG. 6). However, the exact compositions of those culture supernatant fluids were not defined in the study. Without wishing to be bound by theory, Pseudomonas spp., which is commonly found in the environment, and Arcanobacterium pyogenes, a common inhabitant of domestic and wild animals, are believed to have a common signal that C. jejuni recognizes to activate the transcription of the gene(s) necessary for attachment and biofilm formation.

Pili appear to play a role in biofilm formation. In experiments designed to study C. jejuni biofilms by scanning and transmission electron microscopy, the present inventors observed the presence of peritrichous pili interacting with the surface and interacting cell-to-cell in various C. jejuni isolates. To determine whether the filaments shown were pili and not flagella, a flagella deficient mutant (M129::flaAB) was constructed and assayed for its ability to produce pili. The flagella-deficient mutant produced peritrichous pili, as did its wild type parent.

Quorum sensing signals influence biofilm formation. To assay the possibility that quorum sensing plays a role in C. jejuni biofilms, a luxS mutant was constructed, deficient in production of the quorum sensing signaling molecule Al 2 (33). Al 2 played a significant role in C. jejuni biofilm development, as at 48 and 72 hr time points there was a reduction in biofilm formation for the luxS mutant compared with the wildtype C. jejuni M129 (FIG. 5B). To confirm this observation the luxS mutant was grown in the presence of culture supernatant fluids from wildtype M129 mixed 1:1 with MHB. M129 was grown under conditions favorable to producing quorum-sensing molecules. In the presence of wildtype M129 CSF an increase in the luxS mutant biofilm was observed. The luxS mutant was not defective for growth compared to the wildtype.

Few environmental biofilms contain a single bacterial species, and the structure of a biofilm, with cells in close proximity to one another, lends itself to interspecies signaling. (13, 34). During this study, culture supernatant fluids were prepared from various Gram negative and Gram positive bacteria grown under conditions favorable for expression of quorum sensing molecules. C. jejuni isolate M129 was grown in the presence of these CSFs mixed 1:1 with MHB, which was required to support the growth of C. jejuni. In the presence of Pseudomonas spp. and Arcanobacterium pyogenes BBR1CSFs an increase in biofilm development was observed, while CSFs from Clostridium perfringens and Chromobacterium violaceum had no apparent effect on biofilm development.

The role of virulence gene expression in biofilm formation was studied. To assay the possibility that virulence genes play a role in C. jejuni biofilms, a ciaB and tlyA mutant were constructed, deficient in production of a type three secretion system protein and a gene associated with a colonization and hemolytic activity in H. pylori tlyA. tlyA is an uncharacterized gene in C. jejuni, and therefore the exact role it plays in biofilm formation is not known. ciaB does not appear to play a role in biofilm formation were as tlyA showed a slight increase.

Multiple C. jejuni isolates form biofilms. The ability of clinical and non pathogenic C. jejuni isolates to form biofilms was determined. Biofilm formation did not appear to correlate to the pathogenicity of the C. jejuni isolate. Isolate S2B was able to form biofilms to a similar degree to M129 and the other human clinical isolates. However, UMC3, a recent human clinical isolate was the poorest biofilm former tested. NCTC11168 has been reported not attach to PS. It is well know that NCTC11168 loses motility on laboratory passage, and the disparate results may reflect such variation in the two isolates.

The ability of clinical and non-pathogenic C. jejuni isolates to form biofilms was determined. Biofilm formation did not appear to correlate to the pathogenicity of C. jejuni isolate. The non-pathogenic isolate S2B was able to form biofilms to a similar degree to M129 and the other clinical isolate. However, strain UMC3, which was isolated from a patient, was the poorest biofilm former tested.

A preliminary experiment was carried out with an immunizing dose, administered subcutaneously, of pilus protein of 0.15 mg per bird at 7 and 17 days of age. At 24 days of age, the chickens were challenged with 5×10⁸ cells of wild type C. jejuni (heterologous to that from which the pilus protein was derived. At 35 days, the chicks were sacrificed and While on average, the control birds had somewhat higher average numbers of viable cells in the ceca, the immunized chicks had lower numbers of cells and 4 of the 19 chicks had at least a 100-fold drop in viable C. jejuni. Without wishing to be bound by theory, the inventors believe that the challenge dose of bacteria was too high to allow for strong apparent protection.

### Discussion

The recognition of Campylobacter spp. as an important human enteric pathogen has only occurred in the last twenty years, and to date, little is known about the pathogenesis and virulence factors of this organism. One of the least understood factors is the ability of C. jejuni to form biofilms on abiotic and on biological surfaces. Identification of environmental factors responsible for the initiation of C. jejuni biofilms is of primary importance in order to study the survival capabilities of this organism outside the host. Inhibition of biofilm formation by this pathogen could potentially prevent the colonization of various domesticated animals for consumption and companionship and the contamination of our waterways that may serve as sources of human infection.

In their natural environments, bacteria are often challenged by environmental stresses including nutrient starvation, osmotic changes, temperature variation and varied oxygen tensions (10; 13; 14; 19; 20; 25; 26). Bacteria are thought to form biofilms in response to environmental changes, so that there is a transition to a sessile lifestyle (14; 19; 22; 24; 28). Other factors affecting biofilm formation include substratum properties, hydrodynamics, conditioning of the substratum and characteristics of the bathing medium (3; 10; 19; 27), all of which play a role in the rate of bacterial attachment and biofilm formation. In some biofilm models changes in ionic strengths and nutrient concentrations influenced the rate at which bacteria attached and formed biofilms on a surface (10; 13; 14). Since environmental factors and content of the media can affect biofilm formation we tested various bathing media conditions for their affect on C. jejuni biofilms. The results obtained indicated that more nutrient rich media did not support optimal biofilm formation, leading to the conclusion that nutrient-poor environments such as those found in water systems may favor the development of C. jejuni biofilms. We also examined the responses of C. jejuni biofilm formation to varying concentrations of the osmolytes, glucose, sucrose and NaCl: increases in levels of each of these osmolytes caused a noticeable decrease in biofilm formation. Reduced biofilm formation may be the result of a morphological transformation from rod-shaped or spiral cells to coccoid. Coccoid forms may represent a degenerate cell form in which damage to the cell membrane and degradation of cellular components may take place during periods when osmoadaptation is required.

Incubation temperatures and oxygen tension can also influence biofilm formation. However, there have been few studies conducted on the direct effect of aerobiosis on C. jejuni survival and biofilms. In marine and other aquatic environments, dissolved oxygen concentrations may be decreased by lower water flow rates, increased temperature, organic matter and reduced turbulence (6). In keeping with C. jejuni's microaerophilic and thermophilic nature, lower oxygen tensions and higher temperatures favored biofilms, whereas high ambient temperatures, thermophilic temperatures and/or aerobic conditions inhibited biofilm formation.

The physicochemical properties of a surface can affect C. jejuni attachment. C. jejuni was able to attach at varying degrees to hydrophobic and hydrophilic surfaces. Therefore, it seems that C. jejuni has the ability to overcome repulsive forces associated with hydrophobic and hydrophilic surfaces. Some of the factors that may help to overcome these repulsive forces include the presence of flagella and the production of exopolysaccharide (EPS) (10; 12; 14; 24). The hydrophobicity of the bacterial surface can be important in adhesion. Bacteria tend to be negatively charged, but they contain hydrophobic surface components such as flagella that can interact with a surface (10; 30). Studies have shown that treatment of bacteria with protein synthesis inhibitors can markedly decrease biofilm formation and can cause the release of attached bacteria (3; 10; 21). Preincubation of C. jejuni cells with CM inhibited biofilm formation, suggesting that C. jejuni cells synthesize proteins required for attachment and biofilm formation in response to appropriate signals and growth conditions.

Flagella of many bacterial species have a significant role in biofilm formation and the rate of attachment to a surface (10; 21; 22). In this study, C. jejuni flagella minus mutant (M129::flaAB) reduced biofilm formation compared with the wild type strain. The flagella knockout showed a slight reduction in biofilm formation compared to that of wild type at 24 hr, but at 48 and 72 hr time points the biofilm was markedly decreased. These findings may suggest that C. jejuni flagella may be required for more for biofilm development than for attachment to a surface.

Quorum sensing or cell to cell signaling plays a role in cell attachment and detachment to and from biofilms (10; 11; 29). C. jejuni M129 was grown in the presence of bacterial culture supernatant fluids, some were believed to contain quorum sensing signals or autoinducers. Several of the fluids studied are believed to contain a common signal recognized by C. jejuni Al-2 cells because an increase in biofilm formation was observed during the study. To assay the role of quorum sensing in C. jejuni biofilms a mutation was constructed in the luxS gene, which does not allow for the production of Al 2 (11). Quorum sensing in Gram negative bacteria depends on the signaling molecule homoserine lactone (HSL), which controls the expression of several traits including bioluminescence, biofilm formation and virulence factors (11). In C. jejuni, a quorum sensing system that produces the signaling molecule Al 2 has been identified. This system is highly conserved in both Gram positive and Gram negative bacteria and is thought to be used for interspecies communication (11). The luxS gene codes the final enzyme in the biosynthetic pathway for Al-2 production (11). Our studies indicate that biofilm development in C. jejuni requires Al 2 since a reduction in biofilm formation between the luxS mutant and the wild-type was observed. While the exact nature of gene regulation during C. jejuni biofilm formation is not understood, clearly cell to cell communication via Al-2 plays a role in the induction of expression of genes required for these functions.

Biofilm formation by C. jejuni isolates was studied using microscopy techniques and a quantitative staining assay. Biofilm formation did not appear to be correlated with the pathogenesis of the isolate. However, each of the isolates formed a uniform distribution of cells across the polystyrene surface. While there were differences in the density of adherent bacteria, few microcolonies of bacteria were observed during microscopy. The ability of C. jejuni isolates to form biofilms on an abiotic surface may help explain its ability to survive outside it normal host and act as a continuing source of colonization and/or infection contamination for animals and humans.

Despite the environmental limitations of C. jejuni cells, survival in biofilms may play an important role to the transmission of the pathogen to animals and carcasses in husbandry and food processing plants, thus affecting humans. The biofilm variability among isolates could contribute to certain strains being of particular concern for human infections. Our studies should therefore be extended to determine the influence of water distribution systems at these facilities and the correlation of C. jejuni isolates found in biofilms and those that colonize animals and cause human outbreaks. Accordingly, it is important to provide immunogenic compositions for administering to humans and to animals, especially those which are colonized by C. jejuni and which serve as sources of food, milk, water and soil contamination, and thus, human infections.

Abbreviations used herein for amino acids are standard in the art. The abbreviations for amino acid residues as used herein are as follows: A, Ala, alanine; V, Val, valine; L, Leu, leucine; I, Ile, isoleucine; P, Pro, proline; F, Phe, phenylalanine; W, Trp, tryptophan; M, Met, methionine; G, Gly, glycine; S, Ser, serine; T, Thr, threonine; C, Cys, cysteine; Y, Tyr, tyrosine; N, Asn, asparagine; Q, Gln, glutamine; D, Asp, aspartic acid; E, Glu, glutamic acid; K, Lys, lysine; R, Arg, arginine; and H, His, histidine.

As used herein, attenuated means that a bacterial strain is reduced in virulence as compared to a “wild-type” clinical strain that causes disease in a human or particular animal; the attenuated strain does not cause disease in the human or particular animal. Nonlimiting examples of attenuated Campylobacter strains are those which do not express functional fur and/or katA gene products.

With reference to a mutation, functional inactivation of a gene means that there is little or no activity of the gene product. For example, where the gene encodes an enzyme, the encoded product has less than 10%, desirably less than 5% or less than 1% of the enzymatic activity of the product from the wild type gene or there is less than 10%, less than 5% or less than 1% of the expression product. That is to say that the coding sequence can be interrupted with an inserted nucleotide or sequence, partly or wholly deleted or there can be a substitution mutation that changes the amino acid sequence of the encoded protein such that activity is significantly reduced. Alternatively, there can be an insertion, deletion or change in transcription and/or translation regulatory sequences such that expression is reduced or prevented at the level of gene transcription and/or translation of mRNA.

In the present context, Campylobacter biofilm material contains the pilus protein of the present invention, i.e., a pilus protein characterized by the amino acid sequence given in SEQ ID NO:2 or a sequence with at least 90% identity thereto. The biofilm material typically contains extracellular polysaccharide(s) and cells as well. Advantageously the Campylobacter is a C. jejuni or a C. coli. Pilus protein is expressed during the lag and stationary phases of growth and in biofilms. Static growth conditions, for example in Mueller Hinton Medium, favor biofilm formation. The biofilm adheres to the vessel, especially the bottom of the vessel. It can be removed after removal of the spent medium. If the strain of Campylobacter is not attenuated, the viable cells can be killed as known to the art.

The immunogenic compositions and/or vaccines containing the C. jejuni pilus protein of the present invention are formulated by any of the means known in the art. They can be typically prepared as injectables or as formulations for intranasal or oral administration or for administration by oral gavage or ad libitum feeding, for example, in drinking water, either as liquid solutions or suspensions. Solid forms suitable for solution in, or suspension in, liquid prior to injection or other administration may also be prepared. The preparation may also, for example, be emulsified, or the protein(s)/peptide(s) encapsulated in liposomes.

In view of the most likely routes of infection of humans and animals, mucosal immunity is especially advantageous, and the immunogenic compositions advantageously contain an adjuvant such as the nontoxic cholera toxin B subunit (see, e.g., U.S. Pat. No. 5,462,734). Cholera toxin B subunit is commercially available, for example, from the Sigma Chemical Company, St. Louis, Mo. Other suitable adjuvants are available and may be substituted therefor. It is preferred that an adjuvant for an aerosol immunogenic (or vaccine) formulation is able to bind to epithelial cells and stimulate mucosal immunity.

Among the adjuvants suitable for mucosal administration and for stimulating mucosal immunity are organometallopolymers including linear, branched or cross-linked silicones which are bonded at the ends or along the length of the polymers to the particle or its core. Such polysiloxanes can vary in molecular weight from about 400 up to about 1,000,000 daltons; the preferred length range is from about 700 to about 60,000 daltons. Suitable functionalized silicones include (trialkoxysilyl) alkyl-terminated polydialkylsiloxanes and trialkoxysilyl terminated polydialkylsiloxanes, for example, 3 (triethyoxysilyl) propyl terminated polydimethylsiloxane. See U.S. Pat. No. 5,571,531, incorporated by reference herein. Phosphazene polyelectrolytes can also be incorporated into immunogenic compositions for mucosal administration (intranasal, vaginal, rectal, respiratory system by aerosol administration) (See e.g., U.S. Pat. No. 5,562,909).

The active immunogenic ingredients are often mixed with excipients or carriers which are pharmaceutically acceptable and compatible with the active ingredient. Suitable excipients include, but are not limited to, water, saline, dextrose, glycerol, ethanol, or the like and combinations thereof. The concentration of the immunogenic polypeptide in injectable, aerosol or nasal formulations is usually in the range of 0.2 to 5 mg/ml. Similar dosages can be administered to other mucosal surfaces. Such a vaccine can easily be prepared by admixing the protein with a pharmaceutically acceptable carrier. A pharmaceutically acceptable carrier is understood to be a compound that does not adversely effect the health of the animal to be vaccinated, at least not to the extend that the adverse effect is worse than the effects seen when the animal is not vaccinated. A pharmaceutically acceptable carrier can be e.g. sterile water or a sterile physiological salt solution. In a more complex for, the carrier can e.g. be a buffer.

If, however, oral vaccination through drinking water is envisaged, possibly larger amounts of protein have to be given due to spillage of water.

The vaccine according to the present invention may further contain an adjuvant. Immunological adjuvants in general comprise substances that boost the immune response of the host in a nonspecific manner. A number of different adjuvants are known in the art. Examples of adjuvants are Freunds Complete and Incomplete adjuvant, vitamin E, non-ionic block polymers and polyamines such as dextransulphate, carbopol and pyran, bacteria such as Bordetella pertussis or E. coli or bacterium-derived matter, oligopeptide, emulsified paraffin-Emulsigen™ (MVP Labs, Ralston, Nebr.), L80 adjuvant containing aluminum hydroxide (Reheis, N.J.), Quil A (Superphos), or other adjuvants known to the skilled artisan. Also very suitable are surface active substances such as Span, Tween, hexadecylamine, lysolecitin, methoxyhexadecylglycerol and saponins. Furthermore, peptides such as muramyldipeptides, dimethylglycine, tuftsin, are often used. Next to these adjuvants, Immune-stimulating Complexes (ISCOMS), mineral oil e.g. Bayol or Markol, vegetable oils or emulsions thereof and Diluvac.Forte can advantageously be used. The vaccine may also comprise a so-called “vehicle”. A vehicle is a compound to which the polypeptide adheres, without being covalently bound to it. Often used vehicle compounds are e.g. aluminium hydroxide, phosphate, sulphate or oxide; silica; Kaolin or Bentonite. A special form of such a vehicle, in which the antigen is partially embedded in the vehicle, is the so-called ISCOM (EP 109.942, EP 1. The vaccine may also contain preservatives such as sodium azide, thimersol, gentamicin, neomycin, and polymyxin. 80.564, EP 242.380).

Often, the immunogenic composition further comprises stabilizers, e.g. to protect degradation-prone polypeptides from being degraded, to enhance the shelf-life of the vaccine, or to improve freeze-drying efficiency. Useful stabilizers include, without limitation, SPGA, skimmed milk, gelatin, bovine or other serum albumin, carbohydrates e.g. sorbitol, mannitol, trehalose, starch, sucrose, dextran or glucose, proteins such as albumin or casein or degradation products thereof, and buffers, such as alkali metal phosphates. Where an albumin is used, it is desirably from the same species as the animal (or human) to which the immunogenic composition containing it will be administered. Freeze-drying is an efficient method for conservation. Freeze-dried material can be stored stable for many years. Storage temperatures for freeze-dried material may well be above zero degrees, without being detrimental to the material. Freeze-drying can be done according to all well-known standard freeze-drying procedures.

Vaccines comprising the pilus protein, especially a recombinantly expressed pilus protein, are preferably administered mucosally. This can by done by oral administration, through admixing of the vaccine with drinking water or food. Especially for poultry, additional methods such as intraocular vaccination and intranasal vaccination are also very suitable ways of mucosal vaccination.

In addition, if desired, the vaccines may contain minor amounts of auxiliary substances such as wetting or emulsifying agents, pH buffering agents, and/or adjuvants which enhance the effectiveness of the vaccine. Examples of adjuvants which may be effective include but are not limited to: aluminum hydroxide; N-acetyl-muramyl-L-threonyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alanine-2-(1′-2′-dipalmitoyl-sn-glycero-3hydroxyphosphoryloxy)-ethylamine (CGP 19835A, referred to as MTP-PE); and RIBI, which contains three components extracted from bacteria: monophosphoryl lipid A, trehalose dimycolate and cell wall skeleton (MPL+TDM+CWS) in a 2% squalene/Tween 80 emulsion. The effectiveness of an adjuvant may be determined by measuring the amount of antibodies (especially IgA, IgM or IgG) directed against the immunogen resulting from administration of the immunogen in vaccines which comprise the adjuvant in question. Such additional formulations and modes of administration as are known in the art may also be used.

A pilus protein antigen of interest or a peptide derived in sequence from said protein is formulated into vaccines as neutral or salt forms. Pharmaceutically acceptable salts include, but are not limited to, the acid addition salts (formed with free amino groups of the peptide) which are formed with inorganic acids, e.g., hydrochloric acid or phosphoric acids; and organic acids, e.g., acetic, oxalic, tartaric, or maleic acid. Salts formed with the free carboxyl groups may also be derived from inorganic bases, e.g., sodium, potassium, ammonium, calcium, or ferric hydroxides, and organic bases, e.g., isopropylamine, trimethylamine, 2-ethylamino-ethanol, histidine, and procaine.

The immunogenic compositions or vaccines are administered in a manner compatible with the dosage formulation, and in such amount and manner as will be prophylactically and/or therapeutically effective, according to what is known to the art. The quantity to be administered, which is generally in the range of about 100 to 1,000 μg of protein per dose, more generally in the range of about 20 to 1000 μg of protein per dose, depends on the subject to be treated, the capacity of the individual's (or animal's) immune system to synthesize antibodies, and the degree of protection desired. Precise amounts of the active ingredient required to be administered may depend on the judgment of the physician or veterinarian and may be peculiar to each individual, but such a determination is within the skill of such a practitioner.

As an alternative to the use of recombinant pilus protein in immunogenic compositions, one can use killed C. jejuni whole cell preparations from static cultures (for example using Mueller Hinton Broth as the culture medium) by harvesting the biofilm growth in which the pili are expressed from the vessel surface. Where a clinical isolate or a wild type strain is used, desirably the biofilm is treated to kill the bacterial cells within it. Killing of viable cells is well known in the art; non limiting examples of appropriate agents include formalin and BEI. Another approach to the use of whole cell (biofilm) immunogenic compositions is the use of attenuated C. jejuni mutants grow under conditions which result in pilus formation, as in biofilm forming conditions. Pathogenic strains can be attenuated by functionally inactivating the catalase gene (katA, see Day et al. 2000) or the fur gene or mutants lacking one or both of these functions can be isolated. Other species of Campylobacter can be used in place of C. jejuni in the present methods.

Prior to formulating into a vaccine the bacterial cells in the preparation may be inactivated. The bacterial cells may be inactivated using heat (e.g. treatment for two hours at 60° C.) or chemical agents, typically those commonly used for commercial vaccine preparations, following standard procedures known to persons skilled in the art. Chemical agents suitable for inactivating the bacterial preparations of the invention include β-propiolactone (β-Prone, Grand Laboratories Inc., Larchwood, Iowa) or 0.1 M binary-ethyleneinine (BEI). Other methods and materials are well known in the art. Inactivation of the cultures may be confirmed for example, by plating multiple samples onto a suitable solid and incubating the plates under optimal growth conditions.

The vaccine or other immunogenic composition may be given in a single dose; two dose schedule, for example two to eight weeks apart; or a multiple dose schedule or in combination with other vaccines. A multiple dose schedule is one in which a primary course of vaccination may include 1 to 10 or more separate doses, followed by other doses administered at subsequent time intervals as required to maintain and/or reinforce the immune response, e.g., at 1 to 4 months for a second dose, and if needed, a subsequent dose(s) after several months. Humans (or other animals) immunized with the antigen administered according to the present invention are protected from infection by the pathogen from which the antigen of interest is derived.

The methods for the preparation of a vaccine according to the invention need not be complex. In principle, it suffices to raise antibodies to the pilus protein described herein in e.g. an animal, followed by collecting the blood and isolating the antiserum according to standard techniques. Suitable animals for raising such antibodies are e.g. rabbits and chickens. When chickens are used, antibodies can alternatively be obtained from the egg yolk of systemically immunized chickens. In principle the antibodies need not be diluted. They can be given as such, or if necessary even in a concentrated form. Alternatively, if the antibody concentration is very high, the thus obtained antiserum can e.g. be diluted before administration.

It is also possible to obtain cells that produce the desired antibodies, monoclonal antibodies or single chain antibodies of this specificity and to grow these in a fermentor or cell culture apparatus. Antibodies can be harvested afterwards and they can be mixed, if necessary, with a pharmaceutically acceptable carrier. The advantage of such a method is that no animals need to be used for the preparation of the antibodies.

An application of the immunization aspect of the present invention is treating broiler chickens before slaughtering. Such broilers are usually slaughtered at six weeks of age. Therefore, treatment of the animals with an immunogenic composition comprising the pilus protein or a pilus-containing whole cell vaccine, from one to three weeks prior to slaughter causes a significant decrease in the level of Campylobacter contamination.

Antibodies specific to the pilus protein of the present invention can be given as a rather crude preparation, for example by feeding crude antiserum to chickens. Alternative routes of administration include, without limitation, admixing the serum with drinking water or with chicken food. For such purposes, an alternative is freeze-drying of the antibodies, thus enhancing their long term stability, before mixing them with the food or water. Also, the antibodies can be encapsulated before adding them to chicken food. An antiserum or antibody preparation with specificity to the Campylobacter pilus protein of the present invention can be used in the treatment of a patient (especially a human patient) suffering from a Campylobacter infection.

Antibodies specific to the pilus protein of the present invention can be used to detect Campylobacter, especially C. jejuni, in biofilm or fecal samples or for the diagnosis of Campylobacter, especially C. jejuni, infection.

Another use of the pilus protein of the present invention is in the detection of antibodies specific to this protein, for example, to monitor exposure of an animal or human to a Campylobacter, especially C. jejuni, or to monitor the development of an immune response (humoral) to the C. jejuni pilus protein.

Another strategy for immunization of a human or animal is to administer a DNA molecule encoding the pilus protein of the present invention, where the coding sequence is operably linked to transcription and translation sequences appropriate to direct expression in the human or animal into which it has been introduced.

Yet another strategy is to use an avirulent Salmonella strain to express the pilus coding sequence of interest (for prior experiments not involving the pilus protein, see Pawelec et al. (1997) FEMS Immunology and Medical Microbiology 19:137-140). Pawelec et al. suggested that construction of S. typhimurium strains expressing C. jejuni genes encoding a protective C. jejuni antigen such as CjaA, CjaC, LPS, MOMP, flagella or a flagellin subunit, might provide an effective method of obtaining chicken vaccines against both enteropathogens. US Patent Publication 2001/0038844 A1 teaches the expression of a flagellar antigen or fragment in a recombinant Salmonella mutant strain. A number of avirulent strains of Salmonella capable of eliciting mucosal immune responses in chickens have been described. For example, an S. typhimurium delta-cya-delta-crp mutant was developed by Curtiss and Kelly (Curtiss and Kelley. 1987. Infect. Immun. 55:3035-3044) and further modified by Galen and colleagues (Galen et al. 1990. Gene 94:29-35). Also see U.S. Pat. No. 5,424,065 which discloses development of another mutant strain of Salmonella with a mutation in the phoP gene, as well as use of these avirulent mutant organisms as components of vaccines against Salmonella typhimurium. S. typhimurium X3985 Δ-cya-Δ-crp elicited significant protection against rechallenge with a virulent strain of Salmonella in chickens, a response that was shown to be dose and strain dependent (Hassan and Curtiss. 1990. Res. Microbiol. 141:839-850). However, as taught by Curtiss et al., U.S. Pat. No. 5,424,065, successful use of these Salmonella mutants is dependent on the host, the bacterial species, and the route of immunization. Salmonella mutants have also been used as vectors to express foreign antigens from Streptococcus mutants, S. sobrinus (Doggett et al. 1993. Infect. Immun. 61:1859-1966; Jagusztyn-Krynicka et al. 1993. Infect. Immun. 61:1004-1015; Redman et al. 1995. Infect. Immun. 63:2004-2011; Redman et al. 1996. Vaccine 14:868-878), S. equi, Bordetella avium (Gentry-Weeks et al. 1992. J. Bacteriol. 174:7729-7742), B. pertussis, Mycobacterium (Curtiss et al. 1990. Res. Microbiol. 141:797-805), hepatitis B virus (Schodel et al. 1994. Infect. Immun. 62:1669-1676), E. coli heat labile toxin-viral fusion proteins (Smerdou et al. 1996. Virus Res. 41:1-9), and tetanus toxin fragment C (Karem et al. 1995. Infect. Immun. 63:4557-4563).

The amino acids which occur in the various amino acid sequences referred to in the specification have their usual three- and one-letter abbreviations routinely used in the art: A, Ala, Alanine; C, Cys, Cysteine; D, Asp, Aspartic Acid; E, Glu, Glutamic Acid; F, Phe, Phenylalanine; G, Gly, Glycine; H, His, Histidine; I, Ile, Isoleucine; K, Lys, Lysine; L, Leu, Leucine; M, Met, Methionine; N, Asn, Asparagine; P, Pro, Proline; Q, GIn, Glutamine; R, Arg, Arginine; S, Ser, Serine; T, Thr, Threonine; V, Val, Valine; W, Try, Tryptophan; Y, Tyr, Tyrosine.

A protein is considered an isolated protein if it is a protein isolated from a host cell in which it is recombinantly produced. It can be purified or it can simply be free of other proteins and biological materials with which it is associated in nature.

An isolated nucleic acid is a nucleic acid the structure of which is not identical to that of any naturally occurring nucleic acid or to that of any fragment of a naturally occurring genomic nucleic acid spanning more than three separate genes. The term therefore covers, for example, a DNA which has the sequence of part of a naturally occurring genomic DNA molecule but is not flanked by both of the coding or noncoding sequences that flank that part of the molecule in the genome of the organism in which it naturally occurs; a nucleic acid incorporated into a vector or into the genomic DNA of a prokaryote or eukaryote in a manner such that the resulting molecule is not identical to any naturally occurring vector or genomic DNA; a separate molecule such as a cDNA, a genomic fragment, a fragment produced by polymerase chain reaction (PCR), or a restriction fragment; and a recombinant nucleotide sequence that is part of a hybrid gene, i.e., a gene encoding a fusion protein. Specifically excluded from this definition are nucleic acids present in mixtures of DNA molecules, transformed or transfected cells, and cell clones, e.g., as these occur in a DNA library such as a cDNA or genomic DNA library.

As used herein expression directed by a particular sequence is the transcription of an associated downstream sequence. If appropriate and desired for the associated sequence, there the term expression also encompasses translation (protein synthesis) of the transcribed RNA.

In the present context, a promoter is a DNA region which includes sequences sufficient to cause transcription of an associated (downstream) sequence. The promoter may be regulated, i.e., not constitutively acting to cause transcription of the associated sequence. If inducible, there are sequences present which mediate regulation of expression so that the associated sequence is transcribed only when an inducer molecule is present in the medium in or on which the organism is cultivated. In the present context, a transcription regulatory sequence includes a promoter sequence and can further include cis-active sequences for regulated expression of an associated sequence in response to environmental signals, as well known to the art.

One DNA portion or sequence is downstream of second DNA portion or sequence when it is located 3′ of the second sequence. One DNA portion or sequence is upstream of a second DNA portion or sequence when it is located 5′ of that sequence.

One DNA molecule or sequence and another are heterologous to another if the two are not derived from the same ultimate natural source. The sequences may be natural sequences, or at least one sequence can be designed by man, as in the case of a multiple cloning site region. The two sequences can be derived from two different species or one sequence can be produced by chemical synthesis provided that the nucleotide sequence of the synthesized portion was not derived from the same organism as the other sequence.

An isolated or substantially pure nucleic acid molecule or polynucleotide is a polynucleotide which is substantially separated from other polynucleotide sequences which naturally accompany a native transcription regulatory sequence. The term embraces a polynucleotide sequence which has been removed from its naturally occurring environment, and includes recombinant or cloned DNA isolates, chemically synthesized analogues and analogues biologically synthesized by heterologous systems.

A polynucleotide is said to encode a polypeptide if, in its native state or when manipulated by methods known to those skilled in the art, it can be transcribed and/or translated to produce the polypeptide or a fragment thereof. The anti-sense strand of such a polynucleotide is also said to encode the sequence.

A nucleotide sequence is operably linked when it is placed into a functional relationship with another nucleotide sequence. For instance, a promoter is operably linked to a coding sequence if the promoter effects its transcription or expression. Generally, operably linked means that the sequences being linked are contiguous and, where necessary to join two protein coding regions, contiguous and in reading frame. However, it is well known that certain genetic elements, such as enhancers, may be operably linked even at a distance, i.e., even if not contiguous.

The term recombinant polynucleotide refers to a polynucleotide which is made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

Polynucleotide probes include an isolated polynucleotide attached to a label or reporter molecule and may be used to identify and isolate other sequences, for example, those from other strains of Campylobacter jejuni. Probes comprising synthetic oligonucleotides or other polynucleotides may be derived from naturally occurring or recombinant single or double stranded nucleic acids or be chemically synthesized. Polynucleotide probes may be labeled by any of the methods known in the art, e.g., random hexamer labeling, nick translation, or the Klenow fill-in reaction.

Large amounts of the polynucleotides may be produced by replication in a suitable host cell. Natural or synthetic DNA fragments coding for a protein of interest are incorporated into recombinant polynucleotide constructs, typically DNA constructs, capable of introduction into and replication in a prokaryotic or eukaryotic cell, especially Escherichia coli, wherein protein expression is desired. Usually the construct is suitable for replication in a unicellular host, such as E. coli, Bacillus subtilis, Salmonella typhimurium or a Pseudomonas, but a multicellular eukaryotic host may also be appropriate, with or without integration within the genome of the host cell. Eukaryotic host cells include yeast, filamentous fungi, plant, insect, amphibian and avian species. Such factors as ease of manipulation, ability to appropriately glycosylate expressed proteins, degree and control of protein expression, ease of purification of expressed proteins away from cellular contaminants or other factors influence the choice of the host cell.

The polynucleotides may also be produced by chemical synthesis, e.g., by the phosphoramidite method described by Beaucage and Caruthers (1981) Tetra. Letts 22: 1859-1862 or the triester method according to Matteuci et al. (1981) J. Am. Chem. Soc. 103: 3185, and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

DNA constructs prepared for introduction into a prokaryotic or eukaryotic host will typically comprise a replication system (i.e. vector) recognized by the host, including the intended DNA fragment encoding the desired polypeptide, and will preferably also include transcription and translational initiation regulatory sequences operably linked to the polypeptide-encoding segment. Expression systems (expression vectors) may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides may also be included where appropriate from secreted polypeptides of the same or related species, which allow the protein to cross and/or lodge in cell membranes or be secreted from the cell.

An appropriate promoter and other necessary vector sequences will be selected so as to be functional in the host. Examples of workable combinations of cell lines and expression vectors are described in Sambrook et al. (1989) vide infra; Ausubel et al. (Eds.) (1995) Current Protocols in Molecular Biology, Greene Publishing and Wiley Interscience, New York; and Metzger et al. (1988) Nature, 334: 31-36. Many useful vectors for expression in bacteria, yeast, fungal, mammalian, insect, plant or other cells are well known in the art and may be obtained such vendors as Stratagene, New England Biolabs, Promega Biotech, and others. In addition, the construct may be joined to an amplifiable gene (e.g., DHFR) so that multiple copies of the gene may be made. For appropriate enhancer and other expression control sequences, see also Enhancers and Eukaryotic Gene Expression, Cold Spring Harbor Press, N.Y. (1983). While such expression vectors may replicate autonomously, they may less preferably replicate by being inserted into the genome of the host cell.

Expression and cloning vectors advantageously contain a selectable marker, that is, a gene encoding a protein necessary for the survival or growth of a host cell transformed with the vector. Although such a marker gene may be carried on another polynucleotide sequence co-introduced into the host cell, it is most often contained on the cloning vector. Only those host cells into which the marker gene has been introduced will survive and/or grow under selective conditions. Typical selection genes encode proteins that confer resistance to antibiotics or other toxic substances, e.g., ampicillin, neomycin, methotrexate, etc.; complement auxotrophic deficiencies; or supply critical nutrients not available from complex media. The choice of the proper selectable marker will depend on the host cell; appropriate markers for different hosts are known in the art.

Recombinant host cells, in the present context, are those which have been genetically modified to contain an isolated DNA molecule of the instant invention. The DNA can be introduced by any means known to the art which is appropriate for the particular type of cell, including without limitation, transformation, lipofection or electroporation.

As used herein, colonization refers to the establishment of C. jejuni within a host animal or human. In certain animals, for example, poultry (chickens, turkeys, ducks geese and the like) colonization does not result in disease. If colonization is reduced or prevented due to administration of an immunogenic composition comprising the pilus protein of C. jejuni as taught herein, a benefit is that fewer bacteria are introduced into food products and the environment. Infection, in the present context, refers to colonization of a human or animal, with the result that disease symptoms occur.

It is recognized by those skilled in the art that the DNA sequences may vary due to the degeneracy of the genetic code and codon usage. All (synonymous) DNA sequences which code for the pilus protein of interest are included in this invention.

Additionally, it will be recognized by those skilled in the art that allelic variations may occur in the DNA sequences which will not significantly change activity of the amino acid sequences of the peptides which the DNA sequences encode. All such equivalent DNA sequences are included within the scope of this invention and the definition of the regulated promoter region. The skilled artisan will understand that the sequence of the exemplified sequence can be used to identify and isolate additional, nonexemplified nucleotide sequences which are functionally equivalent to the sequences given.

There may be slight modifications in the amino acid sequence of the pilus protein of the present invention. Variation in amino acid sequence may be the result of replacement of one or more amino acids by functional equivalents. Replacement by functional equivalents is often seen. Examples described by Neurath et al (The Proteins, Academic Press, New York (1979), page 14, FIG. 6) are i.a. the replacement of the amino acid alanine by serine; Ala/Ser, or Val/Ile, Asp/Glu, etc. In addition to the variations leading to replacement by functional equivalent amino acids mentioned above, variations may be found, in which an amino acid has been replaced by another amino acid that is not a functional equivalent. This kind of variation only differs from replacement with functional equivalents in that it may yield a protein that has a slight modification in its spatial folding. Both types of variation are often seen in proteins, and they are known as biological variations. It is understood that variations in the amino acid sequence of the pilus protein are allowed to the extent that the immunogenic activity of the polypeptide is retained, i.e., that antibody produced against the variant protein cross reacts with the specifically exemplified pilus protein or the pilus protein of other isolates of C. jejuni.

Hybridization procedures are useful for identifying polynucleotides with sufficient homology to the subject regulatory sequences to be useful as taught herein. The particular hybridization technique is not essential to the subject invention. As improvements are made in hybridization techniques, they can be readily applied by one of ordinary skill in the art.

A probe and sample are combined in a hybridization buffer solution and held at an appropriate temperature until annealing occurs. Thereafter, the membrane is washed free of extraneous materials, leaving the sample and bound probe molecules typically detected and quantified by autoradiography and/or liquid scintillation counting. As is well known in the art, if the probe molecule and nucleic acid sample hybridize by forming a strong non-covalent bond between the two molecules, it can be reasonably assumed that the probe and sample are essentially identical, or completely complementary if the annealing and washing steps are carried out under conditions of high stringency. The probe's detectable label provides a means for determining whether hybridization has occurred.

In the use of the oligonucleotides or polynucleotides as probes, the particular probe is labeled with any suitable label known to those skilled in the art, including radioactive and non-radioactive labels. Typical radioactive labels include ³²P, ³⁵S, or the like. Non-radioactive labels include, for example, ligands such as biotin or thyroxine, as well as enzymes such as hydrolases or peroxidases, or a chemiluminescer such as luciferin, or fluorescent compounds like fluorescein and its derivatives. Alternatively, the probes can be made inherently fluorescent as described in International Application WO 93/16094.

Various degrees of stringency of hybridization can be employed. The more stringent the conditions, the greater the complementarity that is required for duplex formation. Stringency can be controlled by temperature, probe concentration, probe length, ionic strength, time, and the like. Preferably, hybridization is conducted under moderate to high stringency conditions by techniques well know in the art, as described, for example in Keller, G. H., M. M. Manak (1987) DNA Probes, Stockton Press, New York, N.Y., pp. 169-170, hereby incorporated by reference.

As used herein, moderate to high stringency conditions for hybridization are conditions which achieve the same, or about the same, degree of specificity of hybridization as the conditions employed by the current inventors. An example of high stringency conditions are hybridizing at 68° C. in 5×SSC/5× Denhardt's solution/0.1% SDS, and washing in 0.2×SSC/0.1% SDS at room temperature. An example of conditions of moderate stringency are hybridizing at 68° C. in 5×SSC/5× Denhardts solution/0.1% SDS and washing at 42° C. in 3×SSC. The parameters of temperature and salt concentration can be varied to achieve the desired level of sequence identity between probe and target nucleic acid. See, e.g., Sambrook et al. (1989) vide infra or Ausubel et al. (1995) Current Protocols in Molecular Biology, John Wiley & Sons, NY, N.Y., for further guidance on hybridization conditions.

Specifically, hybridization of immobilized DNA in Southern blots with 32P-labeled gene specific probes was performed by standard methods (Maniatis et al.) In general, hybridization and subsequent washes were carried out under moderate to high stringency conditions that allowed for detection of target sequences with homology to the exemplified sequences. For double-stranded DNA gene probes, hybridization can be carried out overnight at 20-25° C. below the melting temperature (Tm) of the DNA hybrid in 6×SSPE 5× Denhardt's solution, 0.1% SDS, 0.1 mg/ml denatured DNA. The melting temperature is described by the following formula (Beltz et al. (1983) Methods of Enzymology, R. Wu, L, Grossman and K Moldave (eds) Academic Press, New York 100:266-285):

Tm=81.5° C.+16.6 Log [Na+]+0.41(+G+C)−0.61 (% formamide)−600/length of duplex in base pairs.

Washes are typically carried out as follows: twice at room temperature for 15 minutes in 1×SSPE, 0.1% SDS (low stringency wash), and once at TM-20° C. for 15 minutes in 0.2×SSPE, 0.1% SDS (moderate stringency wash).

For oligonucleotide probes, hybridization was carried out overnight at 10-20° C. below the melting temperature (Tm) of the hybrid 6×SSPE, 5× Denhardt's solution, 0.1% SDS, 0.1 mg/ml denatured DNA. Tm for oligonucleotide probes was determined by the following formula: TM(° C.)=2(number T/A base pairs+4(number G/C base pairs) (Suggs, S. V et al. (1981) ICB-UCLA Symp. Dev. Biol. Using Purified Genes, D. D. Brown (ed.), Academic Press, New York, 23:683-693).

Washes were typically carried out as follows: twice at room temperature for 15 minutes 1×SSPE, 0.1% SDS (low stringency wash), and once at the hybridization temperature for 15 minutes in 1×SSPE, 0.1% SDS (moderate stringency wash).

In general, salt and/or temperature can be altered to change stringency. With a labeled DNA fragment >70 or so bases in length, the following conditions can be used: Low, 1 or 2×SSPE, room temperature; Low, 1 or 2×SSPE, 42° C.; Moderate, 0.2× or 1×SSPE, 65° C.; and High, 0.1×SSPE, 65° C.

Duplex formation and stability depend on substantial complementarity between the two strands of a hybrid, and, as noted above, a certain degree of mismatch can be tolerated. Therefore, the probe sequences of the subject invention include mutations (both single and multiple), deletions, insertions of the described sequences, and combinations thereof, wherein said mutations, insertions and deletions permit formation of stable hybrids with the target polynucleotide of interest. Mutations, insertions, and deletions can be produced in a given polynucleotide sequence in many ways, and those methods are known to an ordinarily skilled artisan. Other methods may become known in the future.

Thus, mutational, insertional, and deletional variants of the disclosed nucleotide sequences can be readily prepared by methods which are well known to those skilled in the art. These variants can be used in the same manner as the exemplified primer sequences so long as the variants have substantial sequence homology with the original sequence. As used herein, substantial sequence homology refers to homology which is sufficient to enable the variant polynucleotide to function in the same capacity as the polynucleotide from which the probe was derived. Preferably, this homology is greater than 70% or 80%, more preferably, this homology is greater than 85%, even more preferably this homology is greater than 90%, and most preferably, this homology is greater than 95%. All integers between 70 and 100% are also within the scope of this invention. The degree of homology or identity needed for the variant to function in its intended capacity depends upon the intended use of the sequence. It is well within the skill of a person trained in this art to make mutational, insertional, and deletional mutations which are equivalent in function or are designed to improve the function of the sequence or otherwise provide a methodological advantage.

Polymerase Chain Reaction (PCR) is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. (1985) Science 230:1350-1354). Reagents, equipment and instructions are commercially available. By using a thermostable DNA polymerase such as the Taq polymerase, which is isolated from the thermophilic bacterium Thermus aquaticus, the amplification process can be completely automated. Other enzymes which can be used are known to those skilled in the art.

It is well known in the art that the polynucleotide sequences of the present invention can be truncated and/or mutated such that certain of the resulting fragments and/or mutants of the original full-length sequence can retain the desired characteristics of the full-length sequence. A wide variety of restriction enzymes which are suitable for generating fragments from larger nucleic acid molecules are well known. In addition, it is well known that Bal31 exonuclease can be conveniently used for time-controlled limited digestion of DNA. See, for example, Maniatis (1982) Molecular Cloning: A Laboratory Manual, Cold Spring Harbor Laboratory, New York, pages 135-139, incorporated herein by reference. See also Wei et al. (1983 J. Biol. Chem. 258:13006-13512. By use of Bal31 exonuclease (commonly referred to as “erase-a-base” procedures), the ordinarily skilled artisan can remove nucleotides from either or both ends of the subject nucleic acids to generate a wide spectrum of fragments which are functionally equivalent to the subject nucleotide sequences. One of ordinary skill in the art can, in this manner, generate hundreds of fragments of controlled, varying lengths from locations all along the original molecule. The ordinarily skilled artisan can routinely test or screen the generated fragments for their characteristics and determine the utility of the fragments as taught herein. It is also well known that the mutant sequences of the full length sequence, or fragments thereof, can be easily produced with site directed mutagenesis. See, for example, Larionov, O. A. and Nikiforov, V. G. (1982) Genetika 18(3):349-59; Shortle, D, DiMaio, D., and Nathans, D. (1981) Annu. Rev. Genet. 15:265-94; both incorporated herein by reference. The skilled artisan can routinely produce deletion-, insertion-, or substitution-type mutations and identify those resulting mutants which contain the desired characteristics of the full length wild-type sequence, or fragments thereof, i.e., those which express a pilus protein of the present invention or a fragment to which colonization and/or infection inhibiting antibodies are produced in a human or animal to which it is administered.

As used herein percent sequence identity of two nucleic acids is determined using the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:402-410. BLAST nucleotide searches are performed with the NBLAST program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped

BLAST programs, the default parameters of the respective programs (NBLAST and XBLAST) are used. See, for example, the National Center for Biotechnology Information website on the internet.

Monoclonal or polyclonal antibodies, single chain antibodies, human or humanized single chain antibodies or an antigen binding fragment of a human or humanized antibody, specifically binding a C. jejuni pilin protein may be made by methods known in the art. See, e.g., Harlow and Lane (1988) Antibodies: A Laboratory Manual, Cold Spring Harbor Laboratories; Goding (1986) Monoclonal Antibodies: Principles and Practice, 2d ed., Academic Press, New York.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; and Setlow and Hollaender (1979) Genetic Engineering. Principles and Methods, Vols. 1-4, Plenum Press, New York. Abbreviations and nomenclature, where employed, are deemed standard in the field and commonly used in professional journals such as those cited herein.

The invention may be further understood by the following non-limiting examples. These examples are provided for illustrative purposes, and are not intended to limit the scope of the invention as claimed herein. Any variations in the exemplified articles which occur to the skilled artisan are intended to fall within the scope of the present invention.

All references cited herein are hereby incorporated by reference to the extent there is no inconsistency with the present disclosure. The references cited reflect the level of skill in the relevant arts.

### EXAMPLES

### Example 1

### Bacterial Strains and Media

All C. jejuni isolates (Table 1) were maintained on Mueller Hinton agar (Difco) supplemented with 5% bovine blood at 37° C. in a 10% CO₂ incubator.

### Example 2

### Biofilm Formation Assay

C. jejuni isolates to be assayed for biofilm formation were grown overnight in Mueller Hinton broth (MHB) at 37° C. and 10% CO₂ with shaking to an OD₆₀₀ of 0.25. The wells of 24 well polystyrene plates (Corning) containing 1 ml MHB, Brucella A(Difco) or Bolton (Difco) broths were inoculated with overnight cultures of C. jejuni isolates to an OD₆₀₀=0.025 (˜2.5×10⁷ bacteria). Plates were then incubated at 25 or 37° C. in ambient air 10% CO₂ atmosphere for 24, 48 or 72 hr. Following incubation, the medium was removed, the wells were dried for 30 min at 55° C., and 1 ml 0.1% crystal violet (CV) was added for 5 min at room temperature. The unbound CV was removed and the wells were washed twice with H₂O. The wells were dried at 55° C. for 15 min, and bound CV was decolorized with 80% ethanol:20% acetone. 100 μl aliquots of this solution were removed from the wells, placed into a 96 well plate, and the OD₅₇₀ was determined using a micro plate reader (Bio-tek) to determine biofilm formation.

In order to determine the effects of osmolytes on biofilm formation, compounds were added to MHB prior to the biofilm assay. Plates were incubated at 37° in a 10% CO₂ atmosphere for 24 hours before analysis.

### Example 3

### C. jejuni Biofilm Formation on Abiotic Surfaces

Sterile, ˜1×4 cm coupons of acrylonitrile butadiene styrene plastic (ABS), polyvinyl chloride plastic (PVC), polystyrene or copper were placed in 15 ml polypropylene tubes with 5 ml MHB such that the coupons were completely submerged. The tubes were inoculated with C. jejuni to an OD₆₀₀=0.025 and incubated for 24 hr at 37° C. in a 10% CO₂ atmosphere. The coupons were aseptically removed, placed into sterile 15 ml tubes with 2 ml of 0.1M phosphate buffered saline pH 7.3 (PBS) and 20 sterile 4 mm glass beads, and the tubes were vortexed to remove bacterial cells on full speed for 1 min. Viable bacteria were enumerated by dilution plating on Mueller-Hinton agar supplemented with 5% blood.

### Example 4

### Inhibition of Protein Synthesis

Overnight cultures of C. jejuni in MHB were treated with 0.5 μg/ml chloramphenicol (CM) for 15 min at room temperature prior to assaying for biofilm formation in the absence of antibiotic. At this concentration of CM, protein synthesis was inhibited, but the viability of the C. jejuni cells was not impaired. Biofilm formation of CM-treated C. jejuni was assayed as described above.

### Example 5

### Effects of Culture Supernatant Fluids on Biofilm Formation

Gram negative Gram positive bacteria culture supernatant fluids were collected after 24 hr growth in appropriate culture media (Table 2). Cells were removed by centrifugation at 5000 g, and the supernatant culture fluids were filtered through 0.22 μm filters. The supernatant or uninoculated culture medium was mixed 1:1 with MHB. C. jejuni were inoculated in these media, and biofilm formation was assayed as described above.

### Example 6

### Construction of C. jejuni flaAB and luxS Mutants

C. jejuni mutants in genes encoding flagella or the quorum sensing molecule autoinducer 2 (Al 2) were constructed to assess the role of these factors in biofilm formation. The C. jejuni M129 flaA and flaB double mutant was constructed as follows. PCR was used to amplify a 0.9 kb fragment containing the 5′ end of flaA from C. jejuni M129 genomic DNA with primers 5′ CTTTGGCTATCTCGAGACAGGCACTC 3′ (SEQ ID NO:3) and 5′ AAGCTGCAAGCTTGGTGTTAATACGA 3′ (SEQ ID NO:4), while a 0.9 kb fragment containing the 3′ end of flaB was amplified with primers 5′ AAATCAGAGAATTCATTGGTTCGGTG 3′ (SEQ ID NO:5) and 5′ TAACAACAGGATCCTCATAGGTCAGG 3′ (SEQ ID NO:6). The resulting products were digested with XhoI HindIII and EcoRI BamHI, respectively (restriction sites are underlined in the primer sequences). The two fragments were cloned consecutively into the vector pSJB21, which contains a Campylobacter coli aphA 3 kanamycin resistance gene cloned into the HindIII and EcoRI sites of pBC KS, such that the fragments flanked the resistance gene and were oriented in the same direction. This allelic exchange plasmid was introduced into C. jejuni strain M129 by electroporation and putative mutants were selected on Mueller Hinton agar supplemented with 5% blood and 50 μg/ml kanamycin. Allelic replacement was confirmed by PCR and Southern blot. As flaA and flaB are adjacent in C. jejuni, the resulting flaAB mutant contained only the 5′ end of flaA and the 3′ end of flaB, disrupting both genes.

The C. jejuni luxS mutant was constructed similarly, by cloning PCR products derived from the 5′ end of the C. jejuni M129 luxS gene, amplified with primers 5′ CTTCTTGTAACTCGAGTTGTCGTATC 3′ (SEQ ID NO:7) and 5′ AATCAAATAAGCTTATATCATCACCC 3′ (SEQ ID NO:8), and the 3′ end of luxS was amplified with primers 5′ GAACTTAAGAATTCCCAATGCGGAAC 3′ (SEQ ID NO:9) and 5′ ATCTTTATGGGATCCTACGCCTTGAG 3′ (SEQ ID NO:10) into pSJB21. The resulting plasmid was then used for allelic exchange as described above.

### Example 7

### Scanning and Transmission Electron Microscopy

C. jejuni isolates were grown overnight in Mueller Hinton broth (MHB, Difco) at 37° C. and 10% CO₂ with shaking to an optical density at 600 nm (OD₆₀₀) of 0.25. 35 mm plates (Corning) containing sterile polycarbonate membranes and 3 mls of MHB were inoculated with overnight cultures of C. jejuni isolates to an OD₆₀₀=0.025 (˜2.5×10⁷ CFU). Plates were incubated at 37° C. in a 10% CO₂ atmosphere for 24 hr. Following incubation, the polycarbonate membranes containing C. jejuni biofilms were aseptically removed and placed in 4% Formaldehyde 1% Glutaraldehyde in 0.1M phosphate buffered saline (PBS) for fixation. Post fixation membranes were placed in a solution containing 1% osmium tetroxide in deionized H₂O. Membranes were then processed through an EtOH gradient 1×80% 5 mins, 2×95% 5 mins, 2×100% 5 mins, critical point dried; sputter coated with gold, mounted and observed using scanning electron microscopy. Biofilms were also observed through transmission electron microscopy. Cultures were grown as described above. 24-well plates were incubated at 37° C. in a 10% CO₂ atmosphere for 24 hr. Following incubation, 10 μls of C. jejuni biofilm was pipetted onto Formvar coated 400 mesh copper grids for 1 min. Fluid was absorbed using a Whatman filter paper rinsed twice. Following rinsing grids were negatively stained using 1% phosphotungstenic acid. Photographic images were made at various instrumental magnifications to resolve the details of filaments.

### Example 8

### Pilus Harvesting

NCTC strain 11168 is grown in 225 cm cell culture flasks under biofilm forming conditions for 72 hours. Mature biofilms are harvested, pooled and the media removed by centrifugation (30,000×g 30 min). The pelleted biofilm is re-suspended in ethanolamine (0.4M, pH 9.0), and the pili are removed from the cells by mechanical shearing. The sample is then centrifuged (2,500×g, 60 min), and the supernatant containing the solubilized pili is collected. Ammonium sulfate is added to the supernatant to 45% saturation, and the solution is incubated at 4° C. overnight to precipitate the pili. The pili are collected by centrifugation (30,000×g, 30 min) and re-suspended in double distilled H₂O. The crude sample is then viewed using TEM to ensure the presence of fibers in the crude preparation.

### Example 9

### Biofilm Production

1 ml Mueller-Hinton broth is inoculated to an OD₆₀₀ of 0.05 with an overnight broth culture of C. jejuni. Cultures are the TPIGDGPVL n incubated under biofilm forming conditions (37° C., 5% CO₂). At 24, 48 and 72 hour time points, the broth is removed via aspiration, and the biofilm rinsed twice with sterile PBS to remove non-adherent cells and debris. The resultant biofilm is then stained with crystal violet and the amount of biofilm produced measured using a plate reader at 405 nm wavelength.

### Example 10

### Recombinant Pilus Protein Expression and Production

A search of the cj1534c gene was performed using signal 3 P software to determine the presence of any signal sequences in the gene, but none was identified. Without wishing to be bound by theory, the inventors believe that the pilus protein gene of the present invention is a Type III secretory protein.

The entire coding region of gene CJ1534c was cloned into the expression vector pTRC-HIS B (Invitrogen, Carlsbad, Calif.) using standard techniques in E. coli. Specific primers were designed for PCR amplification of the C. jejuni pilus protein gene; forward primer, cj1534F1, AAAAAAAGGAGGATCCCATGTCAGTTAC (SEQ ID NO:11) and reverse primer, cj1534R2, CATAAAGCCCGAATTCTTACATTTTG (SEQ ID NO:12). This strategy introduced a BamH1 site upstream of the coding sequence and an EcoR1 recognition site downstream of the coding sequence. The restriction cut sites were positioned such that the PCR product is cloned in the proper reading frame in the expression plasmid pTRC-HIS B. PCR was performed using the custom primers, and the amplification product was sequenced to ensure the correct gene was amplified. Then the PCR product and the purified pTRC-HIS B vector were digested using Bam H1 and EcoR1. The resultant digestions were cleaned and desalted, then combined and ligated together. Once ligated, the vector containing cj1534c was desalted and electroporated into E. coli DH5α. Colonies containing the plasmid were selected and sequencing was performed to ensure proper plasmid construction.

The resulting plasmid construct was then sequenced to ensure proper insertion of the gene. Upon confirmation of the proper construct, 1 L of LB broth was inoculated to an OD₆₀₀ of 0.05 with the E. coli containing the expression plasmid, and incubated (37° C., 150 RPM) until an OD of 0.8 was reached. IPTG was added to a final concentration of 250 nM, and incubation was continued for an additional 3 hours. The cells were then collected via centrifugation, and the recombinant protein was collected using TALON® affinity purification (BD Biosciences-Clontech, Palo Alto, Calif.) as per the manufacturer's instruction. The collected protein is then concentrated using a protein concentrator (Amincon Corporation, Danvers, Mass.) and a sample was sequenced to ensure proper protein production. Total protein production is determined using a BCA assay, and all collected samples are adjusted to a final concentration of 1 mg/ml with sterile PBS or double distilled H₂O.

### Example 11

### Vaccine Protocol

Commercial broiler chicks are purchased from a commercial hatchery. Upon arrival, a cloceal swab is performed to ensure the birds are free of C. jejuni, and the birds separated into the necessary groups for the experiment. Food and water are available ad libitum. 14 days post hatching, chicks receive the first vaccination and are monitored for adverse side effects. 10 days after the initial vaccination, birds receive a booster vaccination and again are observed for side effects. 10 days post booster vaccination, chicks are challenged with approximately 5×10⁴ CFU C. jejuni via oral gavage. 5 days post challenge, positive controls are swabbed to ensure shedding of C. jejuni and viable cell counts were determined. 10 days post challenge, birds are humanely sacrificed, ceca are collected and cecal contents are removed, serially diluted and plated to enumerate levels of colonization. In addition, ceca and intestines are examined grossly to ensure no non-specific lesion formation has occurred.

### Example 12

### Liposome Production

Commercial liposome preparations are purchased (Sigma, St. Louis, Mo.) and used according to manufacturer specifications. Recombinant protein is used at a concentration of 5 mg/ml for liposome production. Upon completion of liposome formulation, the volume is increased to 1 ml per bird, and administered via oral gavage. The dose of pilus protein is about 0.010 to about 0.500 mg, desirably about 0.250 mg/bird.

### Example 13

### CT Adjuvant

Commercial cholera toxin adjuvant is purchased (Sigma) and diluted with recombinant protein to a final amount of 0.033 μg toxin/bird. This is then diluted to a final volume of 1 ml and administered via oral gavage.

### Example 14

### Colonization of Chickens is Greater by Piliated C. jejuni than by Nonpiliated Strains

A pilus protein knockout mutant of C. jejuni strain NCTC 11168 was produced. This mutation results in the lack of expression of pili on the C. jejuni cell surface as determined by electron microscopy. The pilus protein gene of the present invention is required for expression of the pilus protein, which is 18 kD. The amino acid sequence is provided in SEQ ID NO:2 and in Table 4.

The nonpiliated mutant strain was introduced into two week old chicks, and in parallel experiments, the wild type strain NCTC 11168 was introduced into other two week old chicks. An overall reduction (at least 1000-fold) was observed in the chicks which received the nonpiliated mutant strain. All chicks into which the wild type strain was introduced were colonized (14/14). For those chicks challenged with the nonpiliated mutant, only 5/14 were colonized. This indicates that a mutation in the Cj 1534 gene (coding sequence, SEQ ID NO:1) reduces the ability of the mutant to colonize chickens. Immunogenic compositions comprising this protein are effective for reducing colonization and/or infection of humans and animals by C. jejuni. Reduced colonization of agricultural animals will improve the microbial quality of food products and reduce environmental contamination with C. jejuni. Humans to whom such immunogenic compositions have been administered will have reduced incidence and/or severity of disease caused by C. jejuni.

### Example 15

### Elisa for Antibody Determination

96 well microtiter plates are coated with the appropriate antigen (either recombinant or native protein) overnight. Non-adherent antigen is rinsed off the plates, and the primary antibody, derived from the vaccinated animal, is serially diluted (2-fold) with 1% Fetal Bovine Serum (FBS) and added to the microtiter plate. After incubating overnight, non-adherent primary antibodies are rinsed off the plate, and a secondary antibody diluted 1:400 with 1% FBS is added. The secondary antibodies are commercially available (KPL) and recognize antibodies produced from various animal species (including chicken), and are conjugated with an enzyme. After 4 hours incubation, plates are rinsed and a chromogenic substrate is added to each well. Where the antibody of interest is bound in the well to the antigen coating, the enzyme linked to the secondary antibody cleaves the substrate, resulting in a color development, which is proportional to the level of antibodies present, allowing quantification of bound antibody of interest or this system can serve in a qualitative assay.

Antisera generated in response to the recombinant pilus protein recognized the recombinant pilus protein as well as the native C. jejuni pilus. When serum prepared from chicks vaccinated sub Q with recombinant pilus protein was analyzed, it was found to react with both recombinant and biofilm pilus protein antigen. Antibodies (IgA) from fecal material from chicks vaccinated with liposomes containing recombinant pilus protein reacted with recombinant pilus (3-fold increase over the reaction of nonvaccinated chicks).

### Example 16

### Differential Gene Expression

C. jejuni genes differentially expressed in the chick model and in a 24-hr biofilm to those of C. jejuni genes expressed on plates were identified, and comparisons were made for genes that were upregulated. Two 15 day-old-broiler chickens were infected with 10⁷ C. jejuni by oral inoculation and the cecal contents were collected on 10 days post-infection for the extraction of C. jejuni RNA. Mueller-Hinton broth was inoculated with C. jejuni and the RNA extracted from the 24-hr biofilm. The in vivo obtained RNA and RNA extracted from plate-grown cells were subjected to cDNA synthesis and labeling. Hybridization experiments were performed on Combimatrix DNA microarray slides. A Combimatrix Microarray Imager was utilized for data extraction and the data was analyzed for differential expression using GeneSpring software.

Two slides were used for the hybridization for each animal model with each slide consisting of 6,000 unique probes (in duplicate) to total 12,000 probes per array. The dyes (Cy3, Cy5) representing either principal or control data were switched between slides. Median data (vs. mean data) were utilized to minimize sensitivity to outliers. After normalization of the data, four unique probes (for a total of eight probes) per slide that were specific for the cj1534 gene overexpressed an average of 7.0 fold in the chick model (range of 2.1 to 16.4) and an average of 1.8 fold in the 24-hr biofilm (range 1.3 to 2.2). This data supports that the cjl 534 gene is playing a role in colonization of the avian host.

### Example 17

### Elisa for Antibody Determination

96 well microtiter plates are coated with the appropriate antigen (either recombinant or native protein) overnight. Non-adherent antigen is rinsed off the plates, and the primary antibody, derived from the vaccinated animal, is serially diluted (2-fold) with 1% Fetal Bovine Serum (FBS) and added to the microtiter plate. After incubating overnight, non-adherent primary antibodies are rinsed off the plate, and a secondary antibody diluted 1:400 with 1% FBS is added. The secondary antibodies are commercially available (KPL) and recognize antibodies produced from various animal species (including chicken), and are conjugated with an enzyme. After 4 hours incubation, plates are rinsed and a chromogenic substrate is added to each well. Where the antibody of interest is bound in the well to the antigen coating, the enzyme linked to the secondary antibody cleaves the substrate, resulting in a color development, which is proportional to the level of antibodies present, allowing quantification of bound antibody of interest or this system can serve in a qualitative assay.

Antisera generated in response to the recombinant pilus protein recognized the recombinant pilus protein as well as the native C. jejuni pilus. When serum prepared from chicks vaccinated subcutaneously with recombinant pilus protein was analyzed, it was found to react with both the recombinant pilus protein and biofilm pilus protein antigen. Vaccinated chicks challenged with a high dose of C. jejuni (about 10⁸) had an average of 25% fewer colonized bacteria than control chicks. Notably, a subset of the vaccinated chicks had up to one-four orders of magnitude fewer colonized C. jejuni, although some of the vaccinated chicks had numbers similar to those of the controls. Without wishing to be bound by theory, it is believed that the challenge dose was too high to properly predict results of natural infection.

Antibodies (IgA) from fecal material from chicks vaccinated with liposomes containing recombinant pilus protein reacted with recombinant pilus (3-fold increase over the reaction of nonvaccinated chicks).

Although the description herein contains certain specific information and examples, these should not be construed as limiting the scope of the invention but as merely providing illustrations of some of the presently preferred embodiments of the invention. For example, thus the scope of the invention should be determined by the appended claims and their equivalents, rather than by the examples given.

TABLE 1
C. jejuni isolates used in this study.

| | | Reference |
|---|---|---|
| Strain | Characteristics | or source |
| F38011 | Human clinical isolate | (39) |
| M129 | Human clinical isolate | (12) |
| NCTC11168 | Type strain; genome | (28) |
| | sequence has been | |
| | determined | |
| S2B | Chicken normal flora | L. A. Joens' laboratory |
| | isolate | |
| UMC3 | Human clinical isolate | University of Arizona Medical |
| | | Center, Tucson, AZ |

TABLE 2
Bacteria and culture conditions used for production of Culture Supernatant Fluids.

| | | | Reference |
|---|---|---|---|
| Bacterial species | Strain | Culture conditions | or source |
| Arcanobacterium | BBR1 | Trypticase soy broth (TSB, Difco) supplemented with | (4) |
| pyogenes | | 5% newborn calf serum (Omega Scientific), 37° C. | |
| | | aerobically | |
| Clostridium | Strain 13 | TBS supplemented with 0.5% yeast extract (Difco) | (35) |
| perfringens | | and 0.05% cysteine, 37° C., anaerobically | |
| Pseudomonas | 9027 | MHB, 37° C., aerobically | ATCC^{a} |
| aeruginosa | | | |
| Pseudomonas | PF-5 | MHB, 37° C., aerobically | (10) |
| fluorescens | | | |
| Chromobacterium | CV206 | MHB, 37° C., aerobically | (37) |
| violaceum | | | |

TABLE 3
Nucleotide sequence encoding C. jejuni pilus protein (SEQ ID NO:1)

| | |
|---|---|
| 1 | atgtcagtta caaaacaatt attacaaatg caagcagatg ctcatcattt atgggttaaa |
| 61 | tttcataatt atcactggaa tgtaaaaggt ttgcaatttt tttctataca cgagtacaca |
| 121 | gaaaaagctt atgaagaaat ggcagaactt tttgatagtt gtgctgaaag agttttacaa |
| 181 | cttggcgaaa aagctatcac ttgccaaaaa gttttaatgg aaaatgcaaa aagtccaaaa |
| 241 | gttgcaaaag attgctttac tccgcttgaa gtcatagaac tgatcaaaca agattatgaa |
| 301 | tatcttttag cagaatttaa aaaactcaat gaagcagcag aaaaagaaag tgatactaca |
| 361 | acagctgctt ttgcacaaga aaatatcgca aaatatgaaa aaagtctttg gatgataggc |
| 421 | gctactttac aaggtgcttg caaaatgtaa |

TABLE 4

Amino acid sequence of C. jejuni pilus protein
(SEQ ID NO:2)

MSVTKQLLQMQADAHHLWVKFHNYHWNVKGLQFFSIHEYTEKAYEEMAEL
FDSCAERVLQLGEKAITCQKVLMENAKSPKVAKDCFTPLEVIELIKQDYE
YLLAEFKKLNEAAEKESDTTTAAFAQENIAKYEKSLWMIGATLQGACKM

TABLE 5
Colonization of two week old broiler chickens with
pilus-deficient mutant and wild type strain NCTC11168; inoculating
dose of about 10⁴ viable cells.

| No. | NCTC | No. | Pilus Mutant-NCTC |
|---|---|---|---|
| B1 | 360,000 | Y1 | 30,000 |
| B2 | 10,000 | Y2 | 400,000 |
| B3 | 10,000 | Y3 | 0 |
| B4 | 230,000,000 | Y4 | 0 |
| B5 | 60,000,000 | Y5 | 0 |
| B6 | 33,000,000 | Y6 | 0 |
| B7 | 100,000,000 | Y7 | 1000 |
| B8 | 10,000 | Y8 | 10,000 |
| B9 | 4,500,000 | Y9 | 3000 |
| B10 | 10,000,000 | Y10 | 0 |
| B11 | 20,000,000 | Y11 | 0 |
| B12 | 4,000,000 | Y12 | 0 |
| B21 | 20,000,000 | G25 | 0 |
| B14 | 4,800,000 | R25 | 0 |
| Average | 3.26 × 10⁷ | Average | 3.17 × 10⁴ |

TABLE 6
Colonization of two week old broiler chickens with
pilus-deficient mutant and wild type strain NCTC11168; inoculating
dose of about 10⁴ viable cells

| Bird ID# | NCTC 11168 Pil⁻ | Bird # | NCTC11168 |
|---|---|---|---|
| 45 | ND | 56 | No data |
| 46 | ND | 57 | 1.0E+03 |
| 47 | ND | 58 | 4.0E+06 |
| 48 | ND | 59 | 1.1E+07 |
| 49 | ND | 60 | 5.8E+08 |
| 50 | ND | 61 | 1.0E+05 |
| 51 | ND | 62 | 2.0E+08 |
| 52 | ND | 63 | 1.0E+03 |
| 53 | ND | 64 | 5.5E+07 |
| 54 | ND | 65 | 3.0E+06 |
| 55 | ND | 66 | 4.5E+08 |

ND = Not detected; detection limit was 2.0E+2

### BIBLIOGRAPHY

1. Aydin, F., H. I. Atabay, and M Akan. 2001. The isolation and characterization of Campylobacter jejuni subsp. jejuni from domestic geese (Anser anser). J. Appl. Microbiol. 90:637-642.
2. Baker, J., M. D. Barton, and J. Lanser. 1999. Campylobacter species in cats and dogs in South Australia. Aust. Vet. J. 77:662-666.
3. Bendinger, B., H. H. Rijnaarts, K. Altendorf, and A. J. Zehnder. 1993. Physicochemical Cell Surface and Adhesive Properties of Coryneform Bacteria Related to the Presence and Chain Length of Mycolic Acids. Appl. Environ. Microbiol. 59:3973-3977.
4. Blaser, M. J., I. D. Berkowitz, F. M. LaForce, J. Cravens, L. B. Reller, and W. L. Wang. 1979. Campylobacter enteritis: clinical and epidemiologic features. Ann. Intern. Med. 91:179-85.
5. Blaser, M. J., E. Sazie, and L. P. Williams Jr. 1987. The influence of immunity on raw milk—associated Campylobacter infection. JAMA 257:43-46.
6. Bokkenheuser, V. D., N. J. Richardson, J. H. Bryner, D. J. Roux, A. B. Schutte, H. J. Koornhof, I. Freiman, and E. Hartman. 1979. Detection of enteric campylobacteriosis in children. J. Clin. Microbiol. 9:227-232.
7. Boucher, S. N., E. R. Slater, A. H. Chamberlain, and M. R. Adams. 1994. Production and viability of coccoid forms of Campylobacter jejuni. J. Appl. Bacteriol. 77:303-307.
8. Buswell, C. M., Y. M. Herlihy, L. M. Lawrence, J. T. McGuiggan, P. D. Marsh, C. W. Keevil, and S. A. Leach. 1998. Extended survival and persistence of Campylobacter spp. in water and aquatic biofilms and their detection by immunofluorescent-antibody and -rRNA staining. Appl. Environ. Microbiol. 64:733-741.
9. Costerton, J. W., Z. Lewandowski, D. E. Caldwell, D. R. Korber, and H. M. Lappin-Scott. 1995. Microbial biofilms. Annu. Rev. Microbiol. 49:711-745.
10. Donlan, R. M. 2002. Biofilms: microbial life on surfaces. Emerg. Infect. Dis. 8:881-90.
11. Elvers, K. T. and S. F. Park. 2002. Quorum sensing in Campylobacter jejuni: detection of a luxS encoded signalling molecule. Microbiology 148:1475-1481.
12. Ferrero, R. L. and A. Lee. 1988. Motility of Campylobacter jejuni in a viscous environment: comparison with conventional rod-shaped bacteria. J. Gen. Microbiol. 134:53-59.
13. Fletcher, M. 1988. Attachment of Pseudomonas fluorescens to glass and influence of electrolytes on bacterium-substratum separation distance. J. Bacteriol. 170:2027-2030.
14. Fletcher, M. and G. I. Loeb. 1979. Influence of Substratum Characteristics on the Attachment of a Marine Pseudomonad to Solid Surfaces. Appl. Environ. Microbiol. 37:67-72.
15. Leung, F. Y., G. O. Littlejohn, and C. Bombardier. 1980. Reiter's syndrome after Campylobacter jejuni enteritis. Arthritis Rheum. 23:948-50.
16. Manninen, K. I., J. F. Prescott, and 1. R. Dohoo. 1982. Pathogenicity of Campylobacter jejuni isolates from animals and humans. Infect. Immun. 38:46-52.
17. Mead, P. S., L. Slutsker, V. Dietz, L. F. McCaig, J. S. Bresee, C. Shapiro, P. M. Griffin, and R. V. Tauxe. 1999. Food-related illness and death in the United States. Emerg. Infect. Dis. 5:607-625.
18. Nachamkin, I., B. M. Allos, and T. Ho. 1998. Campylobacter species and Guillain-Barre syndrome. Clin. Microbiol. Rev. 11:555-567.
19. O'Toole, G., H. B. Kaplan, and R. Kolter. 2000. Biofilm formation as microbial development. Annu. Rev. Microbiol. 54:49-79.
20. O'Toole, G. A., K. A. Gibbs, P. W. Hager, P. V. Phibbs Jr, and R. Kolter. 2000. The global carbon metabolism regulator Crc is a component of a signal transduction pathway required for biofilm development by Pseudomonas aeruginosa. J. Bacteriol. 182:425-431.
21. O'Toole, G. A. and R. Kolter. 1998. Initiation of biofilm formation in Pseudomonas fluorescens WCS365 proceeds via multiple, convergent signalling pathways: a genetic analysis. Mol. Microbiol. 28:449-461.
22. O'Toole, G. A. and R. Kolter. 1998. Flagellar and twitching motility are necessary for Pseudomonas aeruginosa biofilm development. Mol. Microbiol. 30:295-304.
23. Pead, P. J. 1979. Electron microscopy of Campylobacter jejuni. J. Med. Microbiol. 12:383-385.
24. Pringle, J. H. and M. Fletcher. 1983. Influence of Substratum Wettability on Attachment of Freshwater Bacteria to Solid Surfaces. Appl. Environ. Microbiol. 45:811-817.
25. Pringle, J. H. and M. Fletcher. 1986. Influence of substratum hydration and adsorbed macromolecules on bacterial attachment to surfaces. Appl. Environ. Microbiol. 51:1321-1325.
26. Reezal, A., B. McNeil, and J. G. Anderson. 1998. Effect of low-osmolality nutrient media on growth and culturability of Campylobacter species. Appl. Environ. Microbiol. 64:4643-4649.
27. Rijnaarts, H. H., W. Norde, E. J. Bouwer, J. Lyklema, and A. J. Zehnder. 1993. Bacterial Adhesion under Static and Dynamic Conditions. Appl Environ Microbiol 59:3255-3265.
28. Rosenberg, M., E. A. Bayer, J. Delarea, and E. Rosenberg. 1982. Role of Thin Fimbriae in Adherence and Growth of Acinetobacter calcoaceticus RAG-1 on Hexadecane. Appl. Environ. Microbiol. 44:929-937.
29. Xie, H., G. S. Cook, J. W. Costerton, G. Bruce, T. M. Rose, and R. J. Lamont. 2000. Intergeneric communication in dental plaque biofilms. J. Bacteriol. 182:7067-7069.
30. Yoshida, A. and H. K. Kuramitsu. 2002. Multiple Streptococcus mutans Genes Are Involved in Biofilm Formation. Appl. Environ. Microbiol. 68:6283-6291.
31. Zimmer, M., H. Barnhart, U. Idris, and M. D. Lee. 2003. Detection of Campylobacter jejuni strains in the water lines of a commercial broiler house and their relationship to the strains that colonized the chickens. Avian. Dis. 47:101-107.
32. Flemming, H. C., Wingender, J., Griegbe, C., Mayer. Physico chemical properties of biofilms. In: Evans L V, editor. Biofilms: recent advances in their study and control. Amsterdam: Harwood Academic Publishers; 2000; p. 19-34.
33. Bullitt, R., L., Makowski. 1995. Structural polymorphism of bacterial adhesion pili. Nature. 373: 164-167.
34. Griffiths, P. L. 1993. Morphological changes of Campylobacter jejuni growing in liquid culture. Lett. Appl. Microbiol. 17: 152 155.
35. Day, W. A., Jr., J. L. Sajecki, T. M. Pitts, L. A. Joens. 2000. Role of Catalase in Campylobacter jejuni Intracellular Survival. Infect. Immun. 68: 6337-6345.

1. A non-naturally occurring recombinant nucleic acid molecule comprising a sequence encoding the Campylobacter jejuni pilus protein having the amino acid sequence given in SEQ ID NO:2 or an amino acid sequence at least 95% identical thereto.

2. (canceled)

3. The nucleic acid molecule of claim 2, wherein said sequence encoding the pilus protein is given in SEQ ID NO:1.

4. The nucleic acid molecule of claim 1 further comprising vector sequences.

5. A recombinant cell into which the non-naturally occurring nucleic acid molecule of claim 1 has been introduced.

6. (canceled)

7. The bacterial cell of claim 6, wherein said cell is an enteric bacterial cell.

8. The enteric bacterial cell of claim 7, which is a nonpathogenic Salmonella cell.

9. An immunogenic composition comprising a Campylobacter pilus protein characterized by the amino acid sequence given in SEQ ID NO:2 or an amino acid sequence having at least 95% identity thereto and a pharmaceutically acceptable carrier.

10. (canceled)

11. The immunogenic composition of claim 9, wherein said Campylobacter pilus protein is a recombinantly produced Campylobacter jejuni pilus protein.

12. The immunogenic composition of claim 9, wherein said composition comprises Campylobacter biofilm material.

13. The immunogenic composition of claim 12, wherein said Campylobacter biofilm material is a killed cell or an attenuated cell Campylobacter biofilm material.

14. (canceled)

15. The immunogenic composition of claim 13, wherein said attenuated Campylobacter biofilm material is a katA-deficient Campylobacter biofilm material or a fur-deficient Campylobacter biofilm material.

16. The immunogenic composition of claim 9 further comprising an immunological adjuvant.

17. The immunogenic composition of claim 16, wherein said immunological adjuvant comprises a cholera toxin subunit B.

18. An immunogenic composition comprising a DNA vaccine molecule capable of expressing the Campylobacter jejuni pilus protein having the amino acid sequence given in SEQ ID NO:2 or an amino acid sequence having at least 95% identity thereto.

19. (canceled)

20. An antibody which specifically binds a Campylobacter jejuni pilus protein characterized by the amino acid sequence given in SEQ ID NO:2 or an amino acid sequence with at least 95% identity thereto.

21. A method for treating Campylobacter infection comprising administering an therapeutically effective amount of the antibody of claim 20 thereto to a human or animal in need thereof.

22. A method for reducing infection and/or colonization of a human or animal with Campylobacter jejuni, said method comprising the step of administering the immunogenic composition of claim 9 to a human or animal in need thereof.

23. (canceled)

24. The method of claim 21, wherein the composition is administered to a mucosal surface of the human or animal or wherein the compositions is administered orally to the human or animal.

25. The method of claim 22, wherein the composition is administered orally to the human or animal or wherein the composition is administered to a mucosal surface of the human or animal.

26. The method of claim 22, wherein said composition further comprises an immunological adjuvant.

26. A method for recombinantly producing a pilus protein comprising the amino acid sequence of SEQ ID NO:2, said method comprising the step of culturing a recombinant cell into which the nucleic acid molecule of claim 1 has been introduced under conditions where said pilus protein is produced.

27. The method of claim 26, further comprising the step of collecting the pilus protein.

28. A method for detecting the presence of Campylobacter pilus protein characterized by the amino acid sequence given in SEQ ID NO:2 or an amino acid sequence with at least 95% identity thereto, said method comprising the steps of:
(a) providing a sample which might contain Campylobacter pilus protein;
(b) contacting the sample with the antibody of claim 20 under conditions which allow the binding of the antibody with the Campylobacter pilus protein; and
(c) detecting the binding of the antibody to the pilus protein.

29-30. (canceled)

31. The method of claim claim 28, wherein said sample is a cecal, fecal, cloacae, poultry, pork or dairy sample.

32. A method of detecting an antibody which specifically binds to a Campylobacter jejuni pilus protein characterized by the amino acid sequence given in SEQ ID NO:2 or an amino acid sequence with at least 95% identity thereto, said method comprising the steps of:
(a) providing a biological sample which might contain antibody which specifically binds to a Campylobacter jejuni pilus protein characterized by the amino acid sequence given in SEQ ID NO:2 or an amino acid sequence with at least 95% identity thereto;
(b) contacting the sample with the pilus protein under conditions which allow binding of the pilus protein to the antibody; and
(c) detecting binding of the pilus protein to the antibody.

33. (canceled)
